SEQUENCE LISTING (23 sequences)

SEQ ID NO: 1 - DNA, 21 bp, Artificial sequence
  Single strand DNA oligonucleotide
aaattgagcccgcagcctccc

SEQ ID NO: 2 - DNA, 19 bp, Artificial sequence
  Single strand DNA oligonucleotide
agcgatgtggctcggctgg

SEQ ID NO: 3 - DNA, 22 bp, Artificial sequence
  Single strand DNA oligonucleotide
attgacactggcaaaacaatgc

SEQ ID NO: 4 - DNA, 20 bp, Artificial sequence
  Single strand DNA oligonucleotide
tccaacacttcgtggggtcc

SEQ ID NO: 5 - DNA, 23 bp, Artificial sequence
  Single strand DNA oligonucleotide
ttgaaatttgctgagctggtgta

SEQ ID NO: 6 - DNA, 23 bp, Artificial sequence
  Single strand DNA oligonucleotide
agcctgagggaattgatgttgat

SEQ ID NO: 7 - DNA, 19 bp, Artificial sequence
  Single strand DNA oligonucleotide
atctctcacgggggttgtg

SEQ ID NO: 8 - DNA, 18 bp, Artificial sequence
  Single strand DNA oligonucleotide
aggctcgcatcagtgtcc

SEQ ID NO: 9 - DNA, 22 bp, Artificial sequence
  Single strand DNA oligonucleotide
ggaaggaacagaagagtgtcgt

SEQ ID NO: 10 - DNA, 20 bp, Artificial sequence
  Single strand DNA oligonucleotide
tcagctctgagtgggagacc

SEQ ID NO: 11 - DNA, 19 bp, Artificial sequence
  Single strand DNA oligonucleotide
gttgtctcctgcgacttca

SEQ ID NO: 12 - DNA, 19 bp, Artificial sequence
  Single strand DNA oligonucleotide
ggtggtccagggtttctta

SEQ ID NO: 13 - DNA, 24 bp, Artificial sequence
  Single strand DNA oligonucleotide
tgttcgccactgtatccagaagtc

SEQ ID NO: 14 - DNA, 23 bp, Artificial sequence
  Single strand DNA oligonucleotide
ccgctcctctttgtcagggtcta

SEQ ID NO: 15 - DNA, 22 bp, Artificial sequence
  Single strand DNA oligonucleotide
ccatgttctcttggtgaatctg

SEQ ID NO: 16 - DNA, 21 bp, Artificial sequence
  ShASS1 sequence
tacagtggcgaacaaattcac

SEQ ID NO: 17 - DNA, 21 bp, Artificial sequence
  ShASS1 sequence
ttgtagagtgaaagtggagac

SEQ ID NO: 18 - DNA, 21 bp, Artificial sequence
  ShASS1 sequence
attaatgctgaagcctgggag

SEQ ID NO: 19 - DNA, 21 bp, Artificial sequence
  ShASS1 sequence
ttgctttgcatactccatcag

SEQ ID NO: 20 - DNA, 21 bp, Artificial sequence
  ShASS1 sequence
atagccttgttccttcagcca

SEQ ID NO: 21 - DNA, 21 bp, Artificial sequence
  Single strand DNA oligonucleotide
atgatattccttcagcctgag

SEQ ID NO: 22 - DNA, 21 bp, Artificial sequence
  Single strand DNA oligonucleotide
aatgagcatggtaaaggatgg

SEQ ID NO: 23 - DNA, 22 bp, Artificial sequence
  Single strand DNA oligonucleotide
ggaagctgtgtttgaagtgtca

(19) United States
(12) Patent Application Publication
(10) Pub. No.: US 2023/0293530 A1
(43) Pub. Date: Sep. 21, 2023

(54) AGENTS FOR SENSITIZING SOLID TUMORS TO TREATMENT

(71) Applicants: Yeda Research and Development Co. Ltd., Rehovot (IL); The United States of America, as Represented by the Secretary, Department of Health and Human Servic, Bethesda, MD (US)

(72) Inventors: Ayelet EREZ, Moshav Bnei Zion (IL); Rom KESHET, Rehovot (IL); Lital ADLER, Rehovot (IL); Eytan RUPPIN, North Bethesda, MD (US); Lee Joo SANG, Seoul (KR)

(73) Assignees: Yeda Research and Development Co. Ltd., Rehovot (IL); The United States of America, as Represented by the Secretary, Department of Health and Human Servic, Bethesda, MD (US)

(21) Appl. No.: 18/012,260

(22) PCT Filed: Jun. 24, 2020
(86) PCT No.: PCT/IL2020/050708

§ 371 Date: Dec. 22, 2022

(51) Int. Cl. A61K 31/519; A61K 31/235; A61K 31/18; A61P 35/00

(52) U.S. Cl. CPC A61K 31/519; A61K 31/18; A61K 31/235; A61P 35/00

(57) ABSTRACT

An agent which increases pyrimidine to purine ration in a cell without decreasing pyrimidine synthesis for use in a method of treating a solid tumor in a subject in need thereof, optionally in combination with an immune-modulating drug.

### SEQUENCE LISTING STATEMENT

The ASCII file, entitled 82688SequenceListing.txt, created on 22 Jun. 2020, comprising 4,967 bytes, submitted concurrently with the filing of this application is incorporated herein by reference.

### FIELD AND BACKGROUND OF THE INVENTION

The present invention, in some embodiments thereof, relates to agents for sensitizing solid tumors to treatment.

Argininosuccinate synthase (ASS1) catalyzes the formation of argininosuccinate from citrulline and aspartate and is a key component in the liver urea cycle (UC). Outside the liver, ASS1 and its subsequent UC enzyme argininosuccinate lyase (ASL) are expressed to form the citrulline-arginine cycle. This cycle supplies the cellular needs for arginine and its downstream essential metabolites, including polyamines, nitric oxide (NO) and proline in a cell specific manner^{1,2}. Accordingly, ASS1 expression is differentially regulated in different cancers. In many cancer types, ASS1 expression is epigenetically downregulated and is associated with poor patients’ prognosis³⁻⁶. In these cancers, downregulation of ASS1 was previously reported to increase the availability of its substrate aspartate for pyrimidine synthesis, supporting cell proliferation⁷. Furthermore, in many cancers, it has been demonstrated that ASS1 deficiency is part of a more general tumor metabolic urea cycle dysregulation (UCD) which further enhances pyrimidine synthesis^{8,9}. The increase in pyrimidine levels generates a mutational bias favoring pyrimidine that correlates with higher levels of hydrophobic-immunogenic antigens and hence with increased response to immunotherapy⁹. Nevertheless, in several common types of cancer, ASS1 is overexpressed for a yet unknown survival benefit¹⁰⁻¹¹. Interestingly, anecdotal studies have reported that the expression of ASS1 in cancer is induced together with gluconeogenic genes without a known functional interaction¹².

Additional background art includes:

WO2018/167778 and WO2018/167780 disclose that changes in nitrogen composition (urea and pyrimidine synthesis metabolites) in biofluids of cancer patients are indicative of cancer diagnosis and prognosis. Specifically, decreased levels of urea and increased levels of pyrimidine synthesis metabolites, can be used as markers for diagnosing, prognosing and treating cancer. Cancers diagnosed, prognosed and/or monitored accordingly are more susceptible to treatment with immune-modulation therapy in combination with agents that specifically promote pyrimidines to purines nucleotide imbalance with an emphasis on immunosuppressive drugs such as anti folate that reduce purine levels.

### SUMMARY OF THE INVENTION

According to an aspect of some embodiments of the present invention there is provided a method of treating a solid tumor in a subject in need thereof, the method comprising administering to the subject a therapeutically effective amount of an agent which increases pyrimidine to purine ratio in a cell without decreasing pyrimidine synthesis, thereby treating the solid tumor.

According to an aspect of some embodiments of the present invention there is provided a composition comprising a therapeutically effective amount of an agent which increases pyrimidine to purine ratio in a cell without decreasing pyrimidine synthesis for use in the treatment of a solid tumor is a subject in need thereof.

According to some embodiments of the invention, the agent is not anti-folate.

According to some embodiments of the invention, the agent decreases purine synthesis.

According to some embodiments of the invention, the agent comprises mizoribine or derivative thereof.

According to some embodiments of the invention, the agent does not affect purine synthesis.

According to some embodiments of the invention, the agent enhances pyrimidine synthesis.

According to some embodiments of the invention, the agent comprises ethyl protocatechuate (EDHB).

According to some embodiments of the invention, the agent enhances purine cyclization.

According to some embodiments of the invention, the agent is selected from the group consisting of BRL-50481 and Sildenafil (e.g., Viagra®).

According to some embodiments of the invention, the solid tumor is non-responsive to an immune-modulating drug.

According to some embodiments of the invention, the method further comprises administering to the subject an immune-modulating drug.

According to some embodiments of the invention, the subject is treated with an immune-modulating drug.

According to some embodiments of the invention, treatment with the immune-modulating drug is following the treatment with the agent.

According to some embodiments of the invention, the agent is administered prior to the immune-modulating drug.

According to some embodiments of the invention, the solid tumor is metastatic.

According to some embodiments of the invention, the solid tumor is selected from the group consisting of lung cancer, breast cancer and colon cancer.

According to some embodiments of the invention, the lung cancer is non-small cell lung cancer (NSCLC).

According to some embodiments of the invention, the solid tumor is characterized by Purine-Rich mutational Bias (PRB).

According to some embodiments of the invention, the immune-modulating drug is an immune-check point protein modulator.

According to some embodiments of the invention, the immune-check point protein modulator is a PD1 antagonist.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G show that ASS1 supports purine synthesis in cancer. A) Purine mutational bias is significantly higher in high ASS1 samples of TCGA samples of breast, lung and colorectal cancer collectively. B) The purine/pyrimidine ratio is increased upon glucose deprivation in murine breast cancer cells. 4T1 cells were grown in the absence of glucose for 24h (baseline) and 48h and quantified by liquid chromatography-mass spectrometry (LC-MS). Nucleotide levels were measured in µg/ml and corrected for protein concentration. Purine/pyrimidine ratio was calculated by dividing (AMP+GMP) by (UMP+CMP) relative concentrations. C) ASS1 inhibition decreases the purine/pyrimidine ration under glucose deprivation. 4T1 cells were grown in the presence or absence of glucose for 48 h and analysed as in 1B. D) DNA sequencing analysis reveals that glucose deprivation results in a purine mutational bias in 4T1 breast cancer cells. Cells were grown and treated as in 5B. E) Purine synthesis inhibition with mizoribine results in a decreased purine/pyrimidine ratio in murine breast cancer cells. 4T1 cells were grown either with or without 300 µM mizoribine for 24 h. Nucleotide levels were measured by LC-MS. The purine/pyrimidine ratio was calculated as in B. F) DNA sequencing analysis reveals that purine synthesis inhibition by a 24 h treatment with 300 µM mizoribine results in a pyrimidine mutational bias in 4T1 breast cancer cells. Each experiment was conducted with biological and technical replicates and repeated at least three times. G) A549 cells were grown for 48 hr in control medium or supplemented with 1.2 µM Methotrexate (Holland-Moran). Nucleotide levels were measured as described below by LC-MS. Purine/pyrimidine ratio was calculated by dividing (AMP+GMP+ATP+GTP) concentration by (UMP+CMP+TMP+UTP+CTP+TTP) concentration before and after methotrexate. All error bars represent SER. *P<0.05, **P<0.01. Each experiment was conducted with biological and technical replicates and repeated at least three times.

FIGS. 2A-D show that restricting purine synthesis decreases the purine to pyrimidine ratio and inhibits growth of high ASS1 cancer cells. A) Purine/pyrimidine ratio is increased upon nutrient deprivation in human colon cancer cells. MC38 cells were grown in either regular medium or in medium without glucose for 24 h. Nucleotide levels were measured by LC-MS. Purine/pyrimidine ratio was calculated by dividing (AMP+GMP) AUC by (UMP+CMP) AUC. B) 4T1 cells with and without shASS1 were grown in glucose free medium either supplemented or not with 10 mM L-Name for 48 hrs. Nucleotide levels were measured by LC-MS. Purine/pyrimidine ratio was calculated by dividing the sum levels of (AMP+GMP) by the sum levels of (UMP+CMP+TMP). C) Purine synthesis inhibition results in decreased purine/pyrimidine ratio in human colon cancer cells. MC38 cells were grown either with or without 300 µM mizoribine for 24 h. Nucleotide levels were measured by LC-MS. Purine/pyrimidine ratio was calculated as in A. D) Purine synthesis inhibition restricts ASS1-overexpressing cancer cell proliferation. 4T1 (left panel) and MC38 cells (right panel) were grown for 24 h with or without 300 µM mizoribine. Cell viability was measured by crystal violet staining. Each experiment was conducted with biological and technical replicates and repeated at least three times. All error bars represent SER. * P<0.05, ** denotes P<0.01, ***P<0.001.

FIGS. 3A-H show that high ASS1 cancers are more sensitive to purine synthesis inhibition than low ASS1-expressing tumors. A) High ASS1 tumors (median ASS1 expression within top tertile) show significantly lower objective response rate to anti-PD1 therapy across cancer types than low ASS1 tumors. B) High PRB tumors (median PRB levels within bottom tertile) show significantly lower objective response rate to anti-PD1 therapy across cancer types than low PRB tumors (see Methods). C) ASS1 mRNA expression levels and NDNO scores are associated with non-response to anti-PD1 therapy in non-small cell lung cancer (NSCLC) (7 responders and 14 nonresponders). D) Proteomic-based NDNO scores associate with lower hydrophobicity of peptide repertoire (Wilcoxon ranksum P<0.05) in a (CPTAC3) lung adenocarcinoma cohort. NDNO scores were used to determine the top tertile (red; n=36) and bottom tertile (blue; n=36); Proteomic NDNO scores denote the ratio of NNDO scores of the tumor vs the matched normal sample (Methods). Hydrophobicity scores were determined by R library Peptides using the metric from Janin et al², where the hydrophobicity of each sample’s peptide repertoire denotes the mean hydrophobicity of all its peptides (Methods). E) 4T1 breast cancer cells were treated with 300 µM mizoribine for 24 hours and analysed by western for LMP-7 immunoproteasome (Left panel). Quantification of the western is shown on the right panel. F) Purine inhibition promotes response to immunotherapy checkpoint inhibitors: 4T1 cells (either control shGFP cells or shASS1 cells) were treated in vitro with mizoribine before injection into female mice mammary fat pads. Mice (each group n=10) were treated with intraperitoneal injection of either PBS control or 250 mg/mouse a-PD1on days 3,5,7,10,12. Mizoribine was given 300□M in vitro. Tumor size was calliper-measured on the designated days (upper panel) and at sacrifice on day 14 (lower panel, p = 0.0476 using student’s t-test). The experiment was conducted with biological and technical replicates and repeated twice (n=10). G) Autologous CD8+T cells from ICT-Non-responsive patients respond to matching NSCLC PDXs explant following MZ treatment. Human IFNγ was measured using ELISA in wells containing cultured CD8+T-cells and PDX explants from two patients (Pat#1, LEP 19 and Pat#2, LEP 20) with metastatic NSCLC on responsive to anti-PD1. In the supernatants of the explant alone, either treated or not-treated with mizoribine, we did not detect human IFNγ. Similarly, adding autologous CD8+T cells to mizoribine-treated or non-treated explants did not induce detectable IFNγ levels. Treatment with anti-PD1, either nivolumab or pembrolizumab, induced a significant elevation in IFNγ secretion only when T cells were incubated with mizoribine-treated explants. One-way ANOVA was performed among the groups; comparison was done between each line-connected two groups). H) A boxplot showing ASS1 expression levels in the primary tumor samples developed metastasis (red) vs those in samples that did not develop metastasis (blue) (Wilcoxon ranksum P<0.05). NS - non-significant, * denotes P<0.05, ***P<0.001, **** P<0.0001. All error bars represent SER.

FIGS. 4A-B show the identification of ASS1 and PD1 in NSCLC explants and autologous T-Cells A) Immunohistochemistry staining of ASS1 expression is positive in the NSCLC PDXs of both patients. B) The cultured primary CD8⁺ T cells were mostly PD1 positive in both patients previously reported to be resistant to anti-PD1 treatment before or when biopsy and blood samples were taken.

FIG. 5 a suggested schematic summary diagram. ASS1 expression in cancer benefits cancer cells’ survival under limiting glucose conditions via S-nitrosylation of gluconeogenic enzymes and by promoting purine synthesis. Treating tumors expressing ASS1 with purine synthesis inhibitors restricts tumor growth and promotes response to immune checkpoint inhibitors.

FIGS. 6A-C show that Ethyl 3,4 dihydroxybenzoate (EDHB) increases pyrimidine to purine and potentiates efficacy of anti PD-1 treatment in vivo. FIG. 6A - Ethyl 3,4 dihydroxybenzoate (EDHB) treatment increases cellular deoxypyrimidine to deoxypurine ratio. 600,000 4T1 breast cancer cells were plated in 10 cm dishes and incubated with 200 uM EDHB for 8 hours. Nucleotide levels were quantified by liquid chromatography-mass spectrometry (LC-MS). The graph demonstrates an increase in pyrimidine / purine ratio following EDHB treatment. FIG. 6B - Ethyl 3,4 dihydroxybenzoate (EDHB) and anti PD-1 combination treatment reduces tumor weight in-vivo. Female Balb/c mice were inoculated with 4T1 cells into the mammary fat pad. After 4 days of tumor initiation, mice were treated daily with 40 mg/kg EDHB or saline administrated IP. 250 ug of anti PD-1 was administrated IP at days 7, 11, 13 and 15. Tumor weight was measured following sacrifice. FIG. 6C - Treatment did not cause any change in the total weight of the mice.

FIG. 7 shows that Sildenafil Citrate (Viagra®) treatment increases cellular pyrimidine to purine ratio. 600,000 mc-38 mouse colon cancer cells (left panel) and 650,000 LLC mouse lung cancer cells (right panel), were plated in 10 cm dishes and incubated with 100 uM Sildenafil citrate for 24/48 hours respectively. Nucleotide levels were and quantified by liquid chromatography-mass spectrometry (LC-MS).

FIG. 8 shows that Sildenafil Citrate (Viagra) and EDHB treatments increases response to anti-PD1 therapy in PDX of non responsive patients.

PDXs from patients with lung cancer non responsive to anti-PD1 were exposed to no treatment, to mizoribine, or to either EDHB (left panel) or Viagra (right panel). IFN-g response was measured following treatment with anti-PD1.

### DESCRIPTION OF SPECIFIC EMBODIMENTS OF THE INVENTION

The present invention, in some embodiments thereof, relates to agents for sensitizing solid tumors to treatment.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

ASS1 is frequently downregulated in different tumor types to increase the availability of its substrate aspartate for nucleotide synthesis, supporting cell proliferation. However, in several common subsets of cancer, including lung, colon and in high ASS1 expressing breast cancers, ASS1 expression is associated with a poor patients’ prognosis. Deciphering the metabolic gains that high ASS1 expression may provide to such cancers, surmising that such understanding may further advance the ability to treat these cancers.

As is illustrated hereinbelow and in the Examples section which follows, the present inventors have shown that ASS1 expression supports cancer progression by enabling tumors to cope with glucose deficiency and to evade the immune system. To counteract these effects in high ASS1 tumors, it is shown that inhibiting purine synthesis increases pyrimidine to purine ratio, elevates the expression of the immunoproteasome and significantly enhances the response of autologous primary CD8+ T cells to anti-PD1, sensitizing these tumors to checkpoint inhibitors. These results suggest that treating patients non-responsive to immune-modulation and/or with high ASS1 cancers with purine synthesis inhibition is beneficial and may also sensitize them to immune checkpoint inhibition therapy.

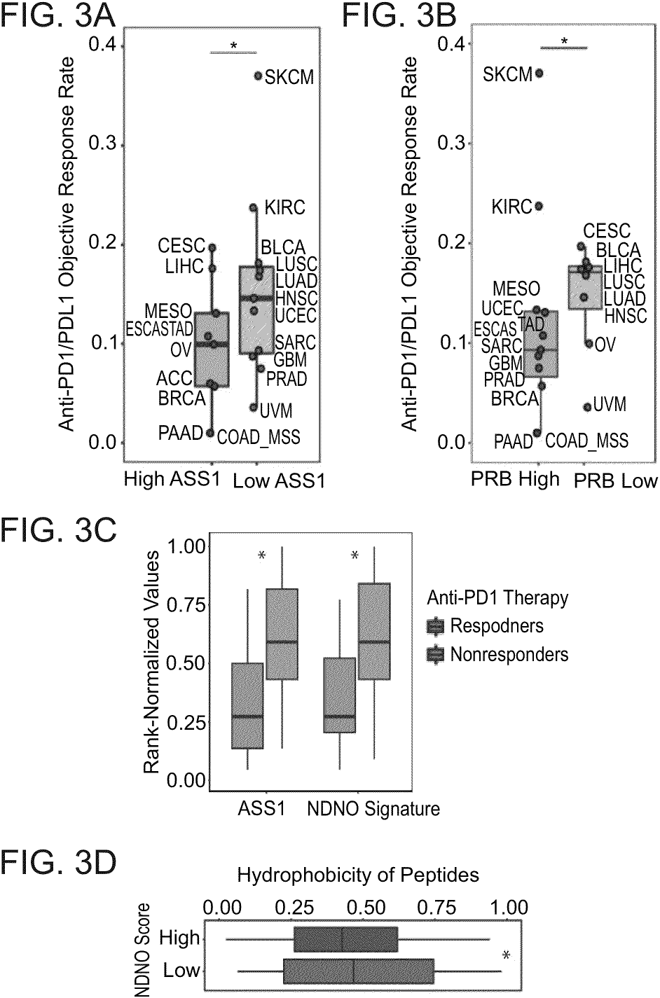

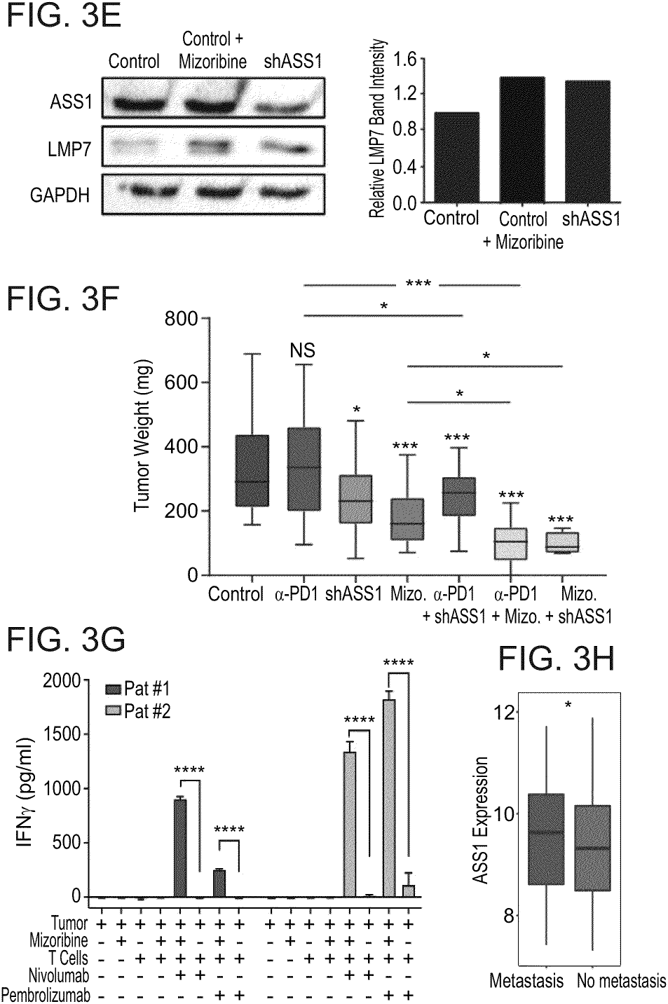

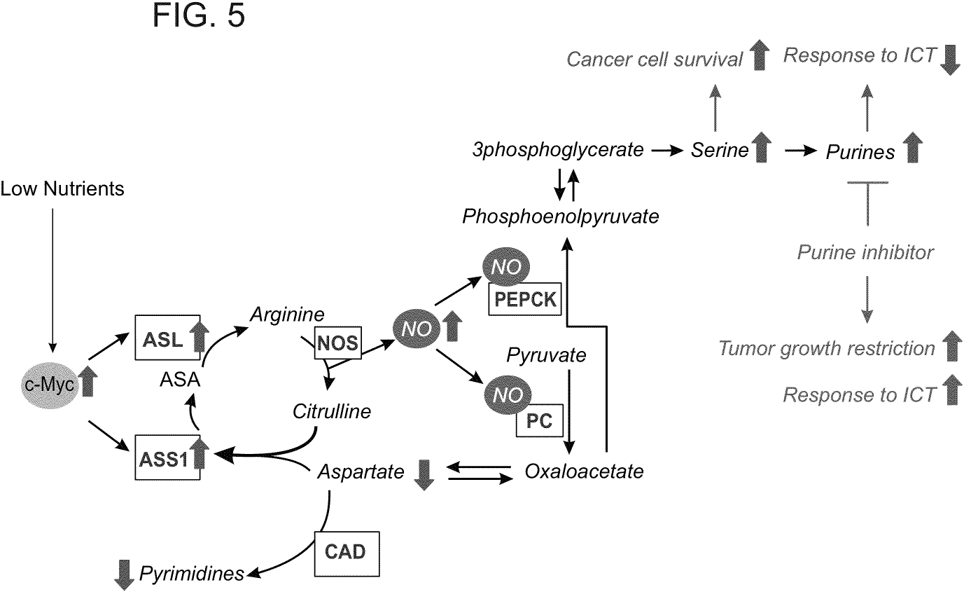

Mizoribine is a compound which exerts its activity through selective inhibition of inosine monophosphate synthetase and guanosine monophosphate synthetase, resulting in inhibition of purine nucleotide synthesis³⁵. The present findings show (FIGS. 3A-H) high cancer killing following mizoribine treatment in high ASS1 breast tumors and suggest that these tumors might be sensitive to purine analogs commonly used in cancer such as mercaptopurine and thioguanine⁴⁷. Beyond that, it shows that high ASS1 expressing tumors respond poorly to ICT and that treatment of these cells with mizoribine, results in the reversal of their PRB signature to a pyrimidine-rich mutational signature, in induction of the immune-proteasome and overall improved response to ICT (FIG. 5). Yet, current purine inhibitors have been reported to have immune-suppressive effects³⁵ and thus, there is a clear need to develop new ways to target purine synthesis that do not contribute to immunosuppression. Consequently, the present inventors have devised an approach that increases the pyrimidine to purine ratio without subjecting the patients to immune-suppression.

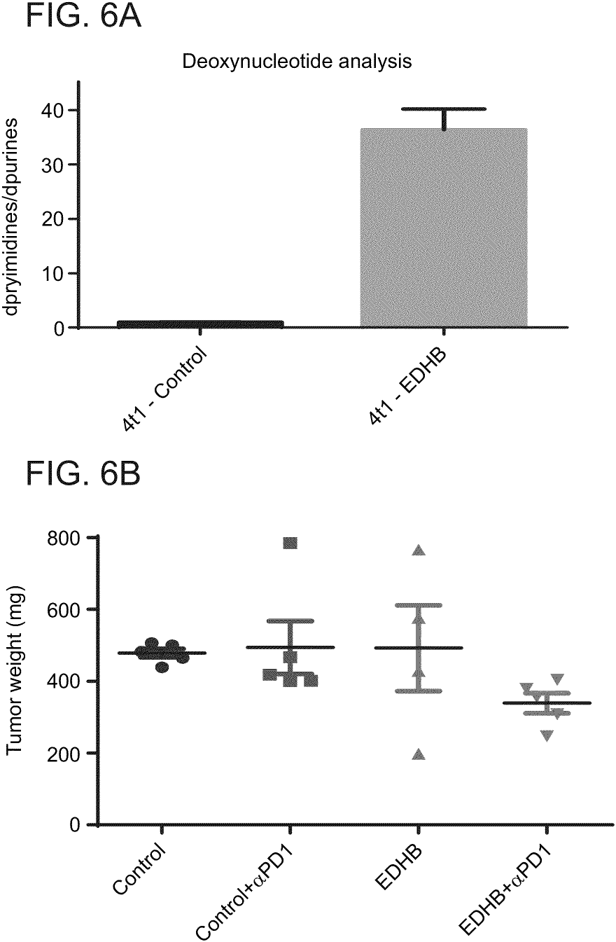

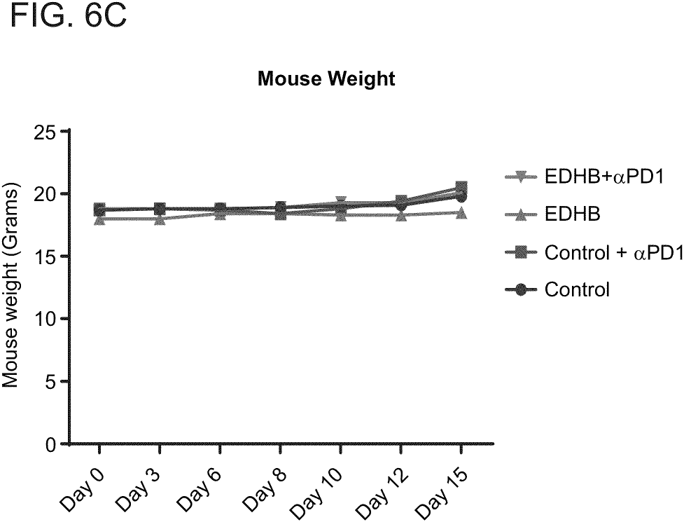

As is shown in FIG. 6A-C to 8 inhibitors which increase overall pyrimidine to purine ratio in the cell without affecting purine synthesis can be effectively used to potentiate treatment with immune-modulators.

Thus, according to an aspect of the invention there is provided a method of treating a solid tumor in a subject in need thereof, the method comprising administering to the subject a therapeutically effective amount of an agent which increases pyrimidine to purine ratio in a cell without decreasing pyrimidine synthesis, thereby treating the solid tumor.

According to an alternative or an additional aspect there is provided a composition comprising a therapeutically effective amount of an agent which increases pyrimidine to purine ratio in a cell without decreasing pyrimidine synthesis for use in the treatment of a solid tumor is a subject in need thereof.

According to an additional or an alternative aspect there is provided a kit comprising a therapeutically effective amount of an immune modulating drug and a therapeutically effective amount of an agent which increases pyrimidine to purine ratio in a cell without decreasing pyrimidine synthesis for use in the treatment of a solid tumor is a subject in need thereof.

The term “treating” refers to inhibiting, preventing or arresting the development of a pathology (in his case cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein the term “subject” refers to a mammal (e.g., human being) at any age or of any gender.

According to specific embodiments, the subject is a human subject.

According to specific embodiments, the subject is diagnosed with a disease (i.e. cancer) or is at risk of developing a disease (i.e., solid tumor cancer).

According to specific embodiments, the subject is not afflicted with an ongoing inflammatory disease (other than cancer).

According to specific embodiments, the subject is not a pregnant female.

According to a specific embodiment, the subject is not responsive to an immune-modulating drug, as further described hereinbelow.

As used herein “solid tumor” (also “cancer”) refers to an abnormal mass of tissue that typically does not contain cysts or liquid areas although tumors that contain such are within the scope of the invention, according to some embodiments. A tumor that is not cancerous is described as “benign” while a cancerous tumor, the targets of this invention, are termed “malignant.” Different types of solid tumors are named for the particular cells that form them, for example, sarcomas formed from connective tissue cells (bone cartilage, fat, etc.), carcinomas formed from epithelial tissue cells (breast, colon, pancreas, etc.) and lymphomas formed from lymphatic tissue cells (lymph nodes, spleen, thymus, etc.). Treatment of all types of solid tumors is within the scope of this invention.

According to specific embodiments, examples of solid tumors include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms’ tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

According to some embodiments, the invention is applicable to sarcomas and epithelial cancers, such as ovarian cancers and breast cancers. In a certain embodiment, the solid tumor cancer is a bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, or any combination thereof.

In another embodiment, the solid tumor is a member of the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the sexual or reproductive organs, Hodgkin’s Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the bladder, cancer of the kidney, renal cell carcinoma, neoplasms of the central nervous system (CNS), neuroectodermal cancer, spinal axis tumors, glioma, meningioma, and pituitary adenoma.

In an embodiment, the solid tumor cancer is ovarian cancer, prostate cancer, breast cancer, cervical cancer, or any combination thereof. In a certain embodiment, the solid tumor cancer is ovarian cancer or another drug-resistant solid tumor. In another embodiment, the solid tumor cancer is ovarian cancer. In another embodiment, the solid tumor cancer is prostate cancer, preferably, androgen-independent prostate cancer. In another embodiment, the solid tumor cancer is breast cancer, preferably, triple-negative breast cancer. In another embodiment, the solid tumor cancer is cervical cancer.

According to a specific embodiment, the solid tumor is selected from the group consisting of lung cancer (e.g., NSCLC). Breast cancer and colon cancer.

According to a specific embodiment, the cancer is refractory to treatment with the immune-modulating drug. According to a specific embodiment, the cancer may be resistant to the treatment from onset of the disease (also referred to as “intrinsic resistance”) or may have acquired resistance during the course of treatment with the immune-modulating drug. While those tumors that are susceptible to treatment are of course within the scope of this invention, it is anticipated that it will be particularly useful in the treatment of refractory tumors.

According to a specific embodiment, the solid tumor is malignant.

According to a specific embodiment, the solid tumor is metastatic.

According to a specific embodiment, the solid tumor is a primary tumor.

According to a specific embodiment, the solid tumor is a metastasis.

According to a specific embodiment, the cancer overexpresses ASS1.

It is suggested that ASS1-overexpressing tumors might have a reverse, purine-rich mutational bias (PRB), characterized by a higher purine to pyrimidine ratio.

As used herein “ASS1” refers to argininosuccinate synthase (EC 6.3.4.5), which catalyzes the formation of argininosuccinate from citrulline and aspartate and is a key component in the liver urea cycle (UC).

According to a specific embodiment, the ASS1 is encoded in human cells by the gene set forth in Accession Number NC_000009.12. Other accession numbers for non-human ASS1 are available from public databases.

As used herein “overexpression” is by at least 5% to almost 1.5 fold expression as compared to a healthy cell from the same tissue, as can be determined at the RNA level from the TCGA database.

Methods of determining gene expression are well known in the art. Typically, gene expression is detected at the protein level or at the nucleic acid level. As shown in the Examples section which follows, ASS1 was measured in various cancer cells using RT-PCR and Western blotting.

### Methods of Detecting the Expression Level of RNA

The expression level of the RNA in the cells of some embodiments of the invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo GJ, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, CA).

Expression level of proteins can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

According to a specific embodiment, the solid tumor is characterized by Purine-Rich mutational Bias (PRB).

As used herein “- purine-rich mutational bias” abbreviated as PRB is characterized by a higher purine to pyrimidine ratio as compared to same in non-cancerous cells of the same origin.

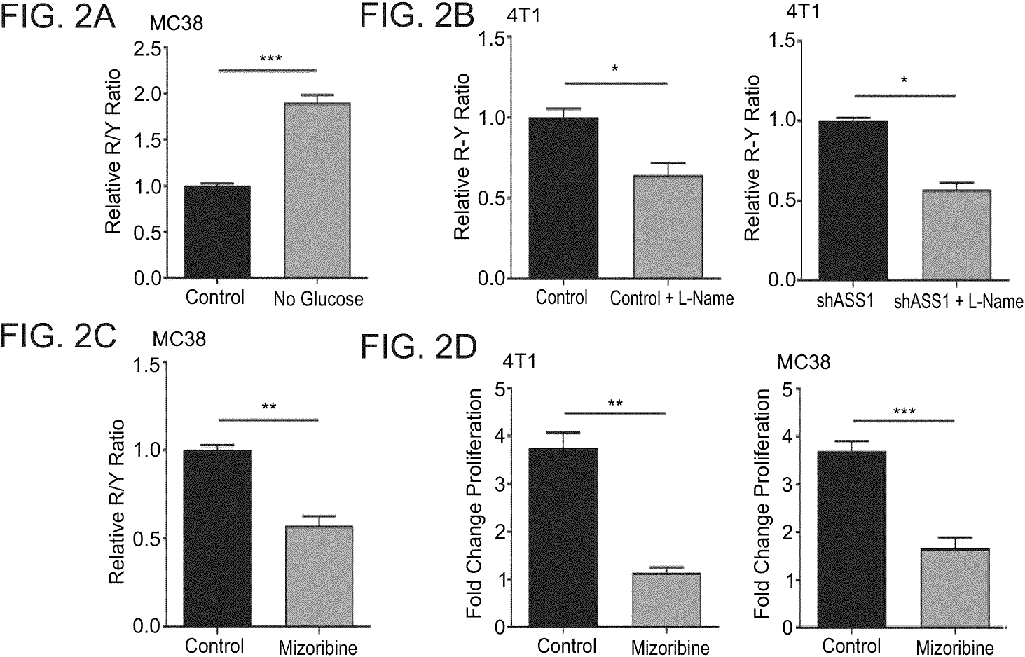

Methods of determining pyrimidine to purine ratio are well known in the art and exemplified in the Examples section which follows (see FIGS. 2A-D). Thus, the levels of these nucleotides and/or mutational bias can be determined by chromatography and mass spectrometry (e.g. LC-MS), whole genome sequencing, DNA sequencing and/or RNA sequencing. According to a specific embodiment the pyrimidine to purine ratio is determined in cancer cells.

As mentioned the present invention contemplates administering to the subject a therapeutically effective amount of an agent which increases pyrimidine to purine ratio in the cell of the solid tumor without decreasing pyrimidine synthesis in the cell.

For example, anti-folates, such as methotrexate, decrease both purine synthesis and pyrimidine synthesis in the cell. It is suggested that the decrease in the synthesis of these metabolic cycles is effected in a sequential manner, whereby decrease in pyrimidine synthesis occurs first and the decrease in purine synthesis follows, leading to a preliminary higher pyrimidine to purine ratio.

This is important since the present inventors have arrived to the conclusion that the increase in the pyrimidine to purine ratio should not compromise purine synthesis. Hence measures should be taken to ensure that the levels of purine synthesis remain unharmed and the level of pyrimidine synthesis can either remain the same (at which case the levels of available purines decreases probably due to physiological derivation/modification of the purines to non-available purines) or even increase. Either case the ratio pyrimidine to purine increases.

Increasing the pyrimidine to purine ratio is expected to increase the numbers of hydrophobic tumor antigens causing better response to immune modulating drugs independent of mutational load.

As used herein “pyrimidine to purine ratio” is calculated by dividing (AMP+GMP) by (UMP+CMP) relative concentrations. AMP and GMP are synthesized from IMP. AMP synthesis involves two enzymes, adenylosuccinate synthetase and adenylosuccinate lyase. GMP synthesis is catalyzed by IMP dehydrogenase and GMP synthetase. OMP decarboxylase is the last enzyme in the pathway for UMP synthesis. CTP synthase catalyzes the formation of CTP from UTP and apyrase converts CTP to CDP and then CMP in two sequential steps.

According to a specific embodiment, increasing purine to pyrimidine ratio ranges between about 1.5-2.5 e.g., about 1.9 for MC38 and about 2.1 fold for 4T1 breast cancer cells.

According to a specific embodiment, the agent decreases purine synthesis.

As used herein “agent” can be interchangeable with drug, medicament, physical treatment and the like.

As used herein “decreases” refers to a decrease in level of the indicated metabolite (e.g., pyrimidine or purine) as compared to same in a cell also referred to as control.

A decrease can be by at least 15%, 20, 20%, 30%, 40%, 50%, 60%, 70%, 80%, 90% or more, say at least 1.5 fold, 2 fold, 2.5 fold, 3 fold, 3.5 fold, 4 fold, 4.5 fold, 5 fold or at least 10 fold.

According to a specific embodiment, an agent which decreases purine synthesis is preferably administered prior to treatment with the immune-modulating drug, to avoid immunosuppression (e.g., about a week before treatment with the immune-modulating drug).

An exemplary agent is mizoribine (also known as “Bredinin”) or derivative thereof.

Shuto et al. 2000 (Journal of the Chemical Society, Perkin Transactions 1 2000, Issue 24, Page 4197 to 4525) teach sugar-modified analogs of mizoribine.

Nair et al. (Molecules 2013, 18, 11576-11585; doi:10.3390/molecules180911576) teach carbocyclic analogs of mizoribine.

Mizoribine analogs are described in PMID: 16438062 which is fully incorporated herein by reference.

Purine inhibitors which can function similarly to mizoribine include, but are not limited to azathioprine and mercaptopurine.

According to another embodiment, the agent does not affect purine synthesis.

As used herein “purine synthesis” refer at least to AMP and GMP. AMP and GMP are synthesized from IMP. AMP synthesis involve two enzymes, adenylosuccinate synthetase and adenylosuccinate lyase. GMP synthesis is catalyzed by IMP dehydrogenase and GMP synthetase.

As used herein “does not affect” refers to an about the same level of the indicated metabolite (e.g., pyrimidine or purine) as compared to the same metabolite in a cancer cell not treated with the agent, but otherwise treated in the same manner as the tumor cell, also referred to as control.

According to an additional or alternative embodiment, the agent enhances pyrimidine synthesis.

As used herein “enhances” or “increases” refers to an increase in level of the indicated metabolite (e.g., pyrimidine or purine) as compared to same in a cancer cell not treated with the agent, but otherwise treated in the same manner as the tumor cell, also referred to as control.

An increase can be by at least 15%, 20, 20%, 30%, 40%, 50%, 60%, 70%, 80%, 90 % or more, say at least 1.5 fold, 2 fold, 2.5 fold, 3 fold, 3.5 fold, 4 fold, 4.5 fold, 5 fold or at least 10 fold, 20 fold, 40 fold, 40 fold, 100 fold.

Such an exemplary agent is ethyl protocatechuate (EDHB). EDHB is a phenolic compound. It can be found in the peanut seed testa. It is also present in wine. It is the ethylic ester of protocatechuic acid.

According to another specific embodiment, the agent is a prolyl 4-hydroxylase inhibitor. Such as taught in WO2012037212A1, which is fully incorporated herein by reference.

According to other specific embodiments, the agent enhances purine cyclization. In doing so, purine synthesis is not affected per se, but purines which are synthesized become unavailable for DNA synthesis. Such purines include cyclic GMP and cyclic AMP.

Examples of such compounds, include, but are not limited to, of BRL-50481 and Sildenafil (e.g., Viagra®).

Typically, other phosphodiesterase (PDE) inhibitors can be used along with the present teachings and are available commercially (see further in Amboss).

TABLE A

| | Active ingredient | Mechanism of Action | |
|---|---|---|---|
| Nonspecific phosphodiesterase inhibitors (inhibitors of PDE3, -4 and -5) | • Theophylline | • Nonspecific PDE inhibition → ↓ hydrolysis of cAMP → ↑ cAMP levels | |
| Phosphodiesterase type 4 inhibitor (PDE4 inhibitor) | • Roflumilast | • PDE4 inhibition → ↑ cAMP in neutrophils, granulocytes, and bronchial epithelium | |
| Phosphodiesterase type 3 inhibitor (PDE3 inhibitor) | • Milrinone • Amrinone | • PDE3 inhibition → ↑ cAMP | |
| | • Cilostazol • Dipyridamole | | ◯ In the myocardium: ↑ cAMP → activation of calcium channels → cardiostimulatory effects (e.g., 1 contractility) |
| | | | ◯ In peripheral vessels: ↑ cAMP → smooth muscle relaxation → vasodilation with reduced cardiovascular preload and afterload |
| | | | ◯ In platelets: ↑ cAMP → inhibition of platelet aggregation |

As mentioned the present inventors identified that when administered with immune-modulating drugs, the agent which increases pyrimidine synthesis can facilitate responsiveness of the tumor to the first.

As used herein “immune-modulating drug” can be a biological molecule (e.g., antibody) or a cell or a small molecule chemical which induces, increases or adds immune activity against the cancer. The immune activity can be either endogenous, i.e., promoting activity of the subject’s immune cells, or exogenous, i.e., adding immune cells against the tumor to the subject.

Immune modulating agents are typically targeting an immune-check point protein.

As used herein the term “immune-check point protein” refers to an antigen independent protein that modulates an immune cell response (i.e. activation or function). Immune-check point proteins can be either co-stimulatory proteins [i.e. positively regulating an immune cell activation or function by transmitting a co-stimulatory secondary signal resulting in activation of an immune cell] or inhibitory proteins (i.e. negatively regulating an immune cell activation or function by transmitting an inhibitory signal resulting in suppressing activity of an immune cell).

Numerous check-point proteins are known in the art and include, but not limited to, PD1, PDL-1, B7H2, B7H3, B7H4, BTLA-4, HVEM, CTLA-4, CD80, CD86, LAG-3, TIM-3, KIR, IDO, CD19, OX40, OX40L, 4-1BB (CD137), 4-1BBL, CD27, CD70, CD40, CD40L, GITR, CD28, ICOS (CD278), ICOSL, VISTA and adenosine A2a receptor.

According to specific embodiments, the immune modulating agent is a PD1 antagonist, such as, but not limited to an anti-PD1 antibody.

PD1 (Programmed Death 1), gene symbol PDCD1, is also known as CD279. According to a specific embodiment, the PD1 protein refers to the human protein, such as provided in the following GenBank Number NP_005009.

Anti-PD1 antibodies suitable for use in the invention can be generated using methods well known in the art. Alternatively, art recognized anti-PD1 antibodies can be used. Examples of anti-PD1 antibodies are disclosed for example in Topalian, et al. NEJM 2012, US Pat. Nos. US 7,488,802; US 8,008,449; US 8,609,089; US 6,808,710; US 7,521,051; and US 8168757, U.S. Pat. Application Publication Nos. US20140227262; US20100151492; US20060210567; and US20060034826 and International Patent Application Publication Nos. WO2008156712; WO2010089411; WO2010036959; WO2011159877; WO2013/019906; WO 2014159562; WO 2011109789; WO 01/14557; WO 2004/004771; and WO 2004/056875, which are hereby incorporated by reference in their entirety.

Specific anti-PD1 antibodies that can be used according to some embodiments of the present invention include, but are not limited to, Nivolumab (also known as MDX1106, BMS-936558, ONO-4538, marketed by BMY as Opdivo); Pembrolizumab (also known as MK-3475, Keytruda, SCH 900475, produced by Merck); Pidilizumab (also known as CT-011, hBAT, hBAT-1, produced by CureTech); AMP-514 (also known as MEDI-0680, produced by AZY and MedImmune); and Humanized antibodies h409Al 1, h409A16 and h409A17, which are described in PCT Pat. Application No. WO2008/156712.

According to specific embodiments, the immune modulating agent is an anti-PD1 antibody. According to specific embodiments, the immune modulating agent is a CTLA4 antagonist, such as, but not limited to an anti-CTLA4 antibody.

CTLA4 (cytotoxic T-lymphocyte-associated protein 4), is also known as CD152. According to a specific embodiment the CTLA-4 protein refers to the human protein, such as provided in the following GenBank Number NP_001032720.

Anti-CTLA4 antibodies suitable for use in the invention can be generated using methods well known in the art. Alternatively, art recognized anti-CTLA4 antibodies can be used. Examples of anti-CTLA4 antibodies are disclosed for example in Hurwitz et al. (1998) Proc. Natl. Acad. Sci. USA 95(17): 10067-10071; Camacho et al. (2004) J. Clin. Oncology 22(145): Abstract No. 2505 (antibody CP-675206); and Mokyr et al. (1998) Cancer Res. 58:5301-5304; U.S. Pat. Nos. US 5,811,097; US 5,855,887; US 6,051,227; US 6,207,157; US 6,207,156; US 6,682,736; US 6,984,720; US 5,977,318; US 7,109,003; US 7,132,281; US 8,993,524 and US 7,605,238, U.S. Pat. Application Publication Nos. 09/644,668; 2005/0201994; 2002/086014, International Application Publication Nos. WO2014066834; WO 01/14424 and WO 00/37504; WO2002/0039581; WO 98/42752; WO 00/37504; WO 2004/035607; and WO 01/14424, and European Patent No. EP1212422B1, which are hereby incorporated by reference in their entirety.

Specific anti-CTLA4 antibodies that can be used according to some embodiments of the present invention include, but are not limited to Ipilimumab (also known as 10D1, MDX-D010), marketed by BMS as Yervoy™; and Tremelimumab, (ticilimumab, CP-675,206, produced by MedImmune and Pfizer).

As mentioned the immune-modulating drug can be a cell, also referred to as adoptive cell therapy (ACT), which typically refers to T cell therapies (which rely on peptide recognition via the T Cell Receptor (TCR), although other cell populations are also included, e.g., NK cells, dendritic cells. T cell therapy can include adoptive T cell therapy, tumor-infiltrating lymphocyte (TIL) immunotherapy, autologous cell therapy, engineered autologous cell therapy (eACT), and allogeneic T cell transplantation. Examples of T cell therapies are described in Rohaan et al. Virchows Archiv (2019) 474:449-461 and U.S. Pat. No. 9,855,298, U.S. Pat. Publication Nos. 2014/0154228 and 2002/0006409, U.S. Pat. No. 5,728,388, and International Publication No. WO 2008/081035.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Pat. Publication No. 2013/0287748, which is herein incorporated by references in its entirety.

The term “engineered Autologous Cell Therapy,” which can be abbreviated as “eACT™” also known as adoptive cell transfer, is a process by which a patient’s own T cells are collected and subsequently genetically altered to recognize and target one or more antigens expressed on the cell surface of one or more specific tumor cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part comprising a costimulatory domain and an activating domain. The costimulatory domain can be derived from, e.g., CD28, and the activating domain can be derived from, e.g., CD3-zeta. In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in U.S. Pat. Publication Nos. 2013/0287748, 2014/0227237, 2014/0099309, and 2014/0050708, and these references are incorporated by reference in their entirety.

The agent which increases pyrimidine to purine ratio, as described herein, can be administered concomitantly to, prior to or following administration of the immune-modulating agent.

According to a specific embodiment, treatment with the immune-modulating agent is following treatment with the agent which increases pyrmidine to purine ratio.

For example, when the agent decreases purine synthesis (e.g., mizoribine) it may be advantageous to treat first with the agent, let the body recover the immune-suppression and only after treat with the immune modulation. An exemplary protocol can be a single daily dose of 150 mg or at a total daily dose of 6-10 mg/kg in a single dose or the divided doses, twice a week (see e.g., Kawasaki et al. 2009 Clin. Dev. Immunol. 681482).

The agents and drugs of some embodiments of the invention can be administered to an organism per se, or in a pharmaceutical compositions (one-when co-formulated, or both when separately administered) where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent which increases pyrimidine to purine ratio (and/or the immune-modulating drug) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington’s Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank’s solution, Ringer’s solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient’s condition. (See e.g., Fingl, et al., 1975, in “The Pharmacological Basis of Therapeutics”, Ch. 1 p.1).

Dosage amount and interval may be adjusted individually to provide effective levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant agents and drug, as exemplified herein, will be developed and the scope of the term “agent” or “drug” is intended to include all such new technologies a priori.

As used herein the term “about” refers to ± 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein.

Other general references are provided throughout this document. The procedures therein are believed to be well known in the art and are provided for the convenience of the reader. All the information contained therein is incorporated herein by reference.

### Example 1

### Materials and Methods

Cell cultures: Patients’ fibroblast studies were performed on anonymized cells devoid of all identifiers. A549, SW620 and 4T1 cells were purchased from NCI60 (RRID: CVCL1381). Control and ASS1-deficient primary fibroblasts were purchased from Coriell Institute for Medical Research. Cells were cultured using standard procedures in a 37° C. humidified incubator with 5% CO2 in Dulbecco’s Modified Eagle’s Medium (DMEM) (Gibco) (for A549 cells and primary fibroblasts) or RPM1 1640 medium (Gibco) (for 4T1, SW620 and MC38 cells) supplemented with 10%-20% heat-inactivated fetal bovine serum, 10% pen-strep and 2 mM glutamine. The cancer cell-lines under glucose-deprivation were grown in a medium containing dialyzed serum and no glucose. The MC38 cell line, derived from mouse colon adenocarcinoma was kindly provided by Dr. Eran Elinav Department of Immunology, The Weizmann Institute of Science. All cells were tested routinely for Mycoplasma using Mycoplasma EZ-PCR test kit (#20-700-20, Biological Industries, Kibbutz Beit Ha’emek).

Virus infection: Cancer cells were infected with either pLKO-based lentiviral vector with short hairpin RNA (shRNA) sequences against either GFP (as a non-target control) or human ASS1 (Dharmacon). Infected cells were selected with 2 µg mL-1 puromycin.

Methotrexate treatment: A549 cells were grown for 48 hr in control medium or supplemented with 1.2 µM Methotrexate (Holland-Moran). Nucleotide levels were measured as described below by LC-MS. Purine/pyrimidine ratio was calculated by dividing (AMP+GMP+ATP+ADP+GTP) concentration by (UMP+CMP+TMP+UTP+CTP+TTP) concentration.

Western blotting: Cells were lysed in RIPA (Sigma-Aldrich) and 1% protease inhibitor cocktail (Calbiochem). Following centrifugation, supernatant was collected and protein content evaluated by the Bradford assay or BCA Protein Assay Kit (ThermoFisher Scientific, cat # 23225). 80 µg from each sample under reducing conditions were loaded into each lane and separated by electrophoresis on a 10% SDS polyacrylamide gel. Following electrophoresis, proteins were transferred to Cellulose Nitrate membranes (Tamar, Jerusalem, Israel). Nonspecific binding was blocked by incubation with TBST (10 mM Tris-HCl (pH 8.0), 150 mM NaCl, 0.1% Tween 20) containing 5% skim milk for 1h at room temperature. Membranes were subsequently incubated with antibodies against: p97 (PA5-22257, Thermo Fisher Scientific), GAPDH (14C10, #2118, Cell Signaling Technology), ASS1 (sc-99178 Santa Cruz Biotechnology), b-Actin (AC-15, Sigma-Aldrich), Tubulin (TU-02, sc-8035, Santa Cruz Biotechnology), PC (ab126707, Abcam), PCK2 (ab137580, Abcam), c-Myc (ab32072, Abcam, kindly provided by prof. zZiv Meshulam and sc-40, Santa Cruz Biotechnology; kindly provided by prof. Yossi Yarden). LMP-7 (cell signaling D1K7X) was kindly provided by Dr. Yifat Marbel). Antibody was detected using peroxidase-conjugated AffiniPure goat anti-rabbit IgG or goat anti-mouse IgG (Jackson ImmunoResearch, West Grove, PA) and enhanced chemiluminescence western blotting detection reagents (EZ-Gel, Biological Industries). Gels were quantified by Gel Doc XR+ (BioRad) and analyzed by ImageLab 5.1 software (BioRad).

RNA processing and quantitative PCR: RNA was extracted from cells by using RNeasy Mini Kit (QIAGENe # 74104). cDNAwas synthesized from 1 µg RNA by using qScript cDNA Synthesis Kit (Quanta #95749). Detection on cDNAs was performed using either SYBR green PCR master mix (Thermo Fisher scientific #4385612) or FastStart Universal Probe Master (Roche #04914058001) with the required primers.

Primer sequences used - Human GAPDH: forward, 5′ -AAATTGAGCCCGCAGCCTCCC-3′ (SEQ ID NO: 1); reverse, 5′ -AGCGATGTGGCTCGGCTGG-3′ (SEQ ID NO: 2); Human HPRT: forward, 5′ -ATTGACACTGGCAAAACAATGC-3′ (SEQ ID NO: 3); reverse, 5′-TCCAACACTTCGTGGGGTCC-3′ (SEQ ID NO: 4); human ASS1 forward, 5′-TTGAAATTTGCTGAGCTGGTGTA-3′ (SEQ ID NO: 5); reverse, 5′-AGCCTGAGGGAATTGATGTTGAT-3′ (SEQ ID NO: 6); Human PHGDH forward, 5′-ATCTCTCACGGGGGTTGTG-3′ (SEQ ID NO: 7); reverse 5′-_AGGCTCGCATCAGTGTCC-3′ (SEQ ID NO: 8); Human PSPH forward 5′-_GGAAGGAACAGAAGAGTGTCGT-3′ (SEQ ID NO: 9); reverse 5′-_TCAGCTCTGAGTGGGAGACC-3′ (SEQ ID NO: 10); Mouse GAPDH: forward, 5′-GTTGTCTCCTGCGACTTCA-3′(SEQ ID NO: 11); reverse, 5′-GGTGGTCCAGGGTTTCTTA-3′; (SEQ ID NO: 12) MouseASS1 F 5′-TGTTCGCCACTGTATCCAGAAGTC-3′ (SEQ ID NO: 13) MouseASS1 F 5′-CCGCTCCTCTTTGTCAGGGTCTA-3 (SEQ ID NO: 14)Human ASL forward, 5′GGAAGCTGTGTTTGAAGTGTCA—3′(SEQ ID NO: 23).;reverse 5′CCATGTTCTCTTGGTGAATCTG-3′ (SEQ ID NO: 15).

In vivo animal studies: Animal experiments were approved by the Weizmann Institute Animal Care and Use Committee Following US National Institute of Health, European Commission and the Israeli guidelines (IACUC 08921218-1 and 38220917-2). Tissue-specific knock out of ASS1 in hepatocytes was generated by crossing ASS1^{f/f} C57BL/6 mice with transgenic mice overexpressing albumin (Alb) Cre.

Syngeneic mouse breast cancer model: 8 weeks old BALB/c female mice were injected with 1×10⁶ 4T1 breast cancer cells in the mammary fat pad. For drug experiments, Mice (each group n=10) were treated with IP injection of either PBS control, 250 mg/mouse a-PD1(clone 29F.1A12, BioCell) given on days 7, 11, 13. Mizoribine treated groups were injected with 4T1 cells that were pre incubated with 300 µM of Mizoribine 48 hrs prior to inoculation. Two weeks following the injection an advanced tumor was resected and measured.

LC-MS analysis: The LC-MS/MS instrument consisting of an Acquity I-class UPLC system (Waters) and Xevo TQ-S triple quadrupole mass spectrometer (Waters), equipped with an electrospray ion source, and operated in positive ion mode was used for analysis of nucleoside monophosphates. MassLynx and TargetLynx software (version 4.1, Waters) were applied for the acquisition and analysis of data. Chromatographic separation was done on a 100 mm × 2.1 mm internal diameter, 1.8-µm UPLC HSS T3 column equipped with 50 mm × 2.1 mm internal diameter, 1.8-µm UPLC HSS T3 pre-column (both Waters Acquity) with mobile phases A (10 mM ammonium acetate and 5 mM ammonium hydrocarbonate buffer, pH 7.65 adjusted with 10% acetic acid) and B (acetonitrile) at a flow rate of 0.3 mL min-1 and column temperature 25° C. A gradient was used as follows: for 0-3 min the column was held at 0.2% B, then 3-3.5 min a linear increase to 100% B, 3.5-4.0 min held at 100% B, 4.0-4.5 min back to 0.2% B and equilibration at 0.2% B for 2.5 min. Samples kept at 8° C. were automatically injected in a volume of 3 µl. For mass spectrometry, argon was used as the collision gas with a flow of 0.10 mL min-1. The capillary voltage was set to 2.50 kV, source temperature 150° C., desolvation temperature 400° C., cone gas flow 150 1 hr-1, desolvation gas flow 800 1 hr-1. Nucleotide concentration was calculated using a standard curve of the relevant nucleotide concentration in each sample. Standard curves included increasing concentration of all measured nucleotides ranging from 0-10 ug/ml that were positioned at the beginning and at the end of each run. All the calculated values for the different nucleotides in each sample fell within the standard curve range. Analytes were detected in positive mode using multiple-reaction monitoring listed in Lee, J. S. et al. 2018 (REF_9).

### Sample Procurement and Generation of PDXs and Patient’s Peripheral CD8+ T Cells

Two patients bearing metastatic non-small cell lung cancer (NSCLC) were included in this study. Blood samples and fresh tumor tissue samples were procured just after their biopsy with patient consent and with Helsinki approval (number 0093-19-SOR). The samples were placed in serum free DMEM (Gibco) media then processed for implantation in NSG (NOD scid gamma) mice. Tumor tissue samples were implanted subcutaneously in dorsal flanks of the mice to form patient-derived xenografts (PDXs). All animal experiments were done under Institutional Animal Care and Use Committee (IACUC) of Ben-Gurion University of the Negev (BGU’s IACUC) according to specified protocols aiming to ensure animal welfare and reduce su □ ering. The Animal ethical clearance protocol number used for this research is IL-80-12-2015. After isolation of peripheral blood mononuclear cells (PBMCs) from the peripheral blood of the patients according to standard protocol using Lymphocyte Separation Medium (LSM, MP Biomedicals, SKU 0850494-CF), CD8⁺ T cells were isolated from PBMCs by employing human CD8 Microbeads (Miltenyi Biotec, 130-045-201), LS column (Miltenyi Biotec, 130-042-401) and MidiMACS Separator (130-042-302) following manufacturer’s protocol. For culturing and maintenance of CD8 T cells, RPMI containing 10% human male AB plasma (Sigma, H4522), 1 mM sodium pyruvate, 2 mM L-glutamine, 0.1 mM MEM non-essential amino acids, 1% penicillin/streptomycin, 10 mM HEPES (Life Technologies), 200 IU/mL recombinant human IL-2 (PeproTech) and 50 ng/mL anti-human CD3 Antibody (BioLegend, 317302) were used for the first 48 hrs followed by culturing and passaging in media containing 200 IU/mL of IL-2.

### Co-Culture of Tumor Tissue Explants With Autologous CD8+ T Cells

2×2×2 mm³ tumor tissue explants were cut from PDXs with a specific tissue cutter and seeded singly in 48 well flat-bottom plates and incubated with either mizoribine (0.3 mM) in 400 µl of media or with only media for 10 hrs. Then, explants were washed once and transferred into new 48 well flat-bottom plates containing 2×10⁵ CD8+T cells/well along with nivolumab (Opdivo, 20 µg/mL), pembrolizumab (Keytruda, 20 µg/mL) or mock solventin fresh 400 µl RPMI containing 20 IU/ml ofIL-2. After 18 hours of incubation, the supernatant was collected from the wells and then assayed for standard IFNγ ELISA assay (ELISA MAX, Biolegend) according to manufacturer’s instructions.

### Immunohistochemistry of PDX Tumors

Five micrometer paraffin embedded tissue sections were de-paraffinized and rehydrated. Endogenous peroxidase was blocked with three percent H₂O₂ in methanol. The antigen retrieval staining for ASS1 was done in citric acid (pH 6), for 10 minutes, followed by 7 minutes in acetone. After pre-incubation with 20% normal horse serum and 0.2% Triton X-100 for 1 hour at RT, biotin block via Avidin/Biotin Blocking Kit (SP-2001, Vector Laboratories, Ca, USA), sections were incubated with the ASS1 antibody (1:100 dilution, Abcam, ab124465, CA, USA). All antibodies were diluted in PBS containing 2% normal horse serum and 0.2% Triton. Sections were incubated overnight at RT followed by 24 h at 4° C. Sections were washed three times in PBS and incubated with secondary biotinylated IgG at RT for 1.5 hour, washed three times in PBS and incubated with avidin-biotin Complex (Elite-ABC kit, Vector Lab) at RT for additional 90 min followed by DAB (Sigma) reaction. Stained sections were examined and photographed by a bright field microscope (E600, Tokyo, Japan) equipped with Plan Fluor objectives (10x) connected to a CCD camera (DS-Fi2, Nikon).

### ASS1 Expression in Tumor vs Healthy Tissue Samples

The cancer genome atlas (TCGA) gene expression and phenotypic information of 9,273 patients (6,228 GTEx healthy tissue samples) was downloaded, encompassing 25 cancer types for which corresponding healthy control samples via UCSC Xena browser (www(dot)xena(dot)ucsc(dot)edu)²:
Gene expression (TOIL DESeq normalized count data):
www(dot)toil(dot)xenahubs(dot)net/download/TcgaTargetGtex_RSEM_Hugo_norm_cou nt(dot)gz, Phenotype information:
www(dot)toil(dot)xenahubs(dot)net/download/TcgaTargetGTEX_phenotype(dot)txt(dot) gz

The expression of ASS1 in these cancer was compared to healthy tissue samples using Wilcoxon rank-sum test.

### TCGA Whole-Exome Sequencing Analysis

For mutation analysis, the mutation data was downloaded from cbioportal (N=11,072), as it integrates the mutation analysis from different TCGA centers to avoid center-specific bias in mutation calls. (For the analyses described in the Methods Section “ASS1 expression in tumor vs healthy tissue samples,” the present inventors used the TCGA data from UCSC Xena browser (www(dot)xena(dot)ucsc(dot)edu) because it includes TCGA RNAseq data normalized together with GTEx healthy tissue samples, which are absent in cbioportal.)

To study the Purine-Rich mutational Bias (PRB) the present inventors consider the fraction of transversions from puRines (R) to pYrimidines (Y); f(R->Y) denotes the ratio of R->Y point mutations to all point mutations on the DNA sense strand in a given sample. The fraction of transversions from pyrimidines to purines (Y->R), is defined in an analogous manner. PRB is defined as the difference between the two fractions, i.e.

$$\text{PRB} = \frac{N \left(Y \to R\right) - N \left(R \to Y\right)}{m u t a t i o n a l l o a d} = f \left(Y \to R\right) - f \left(R \to Y\right) ,$$

where N(R->Y) and N(Y->R) denote the number of R->Y and Y->R single nucleotide polymorphisms (SNPs) on the DNA sense strand, respectively, and ‘mutational load’ is the total number of SNPs in a given sample. PRB was calculated using all single nucleotide variants (SNV). PRB levels were compared for ASS 1-low (bottom tertile) vs ASS1-high (top tertile) samples using Wilcoxon ranksum test for lung, breast, and colorectal cancers collectively.

### NDNO Signature

The present inventors used the gene expression-based nutrient deprivation signature ³ to estimate the extent of cellular response to nutrient deprivation driven by MYC. The nutrient deprivation signature was combined with the expression levels of enzymes involved in S-nitrosylation (ASS1, ASL, NOS1) to construct the nutrient deprivation/NO (NDNO) signature. The mean expression level of the ND signature genes together with ASS1, ASL, and NOS1 was calculated in each sample and then compared to ASS1 expression.

### Association of ASS1 Expression and NDNO Signature With Patients’ Prognosis

Kaplan Meier analysis was performed to identify the association of ASS1 with patient survival. The survival of patients with high ASS1 samples (top tertile) vs the remainder of the samples was compared using a logrank test ⁴. An analogous analysis was performed for the samples that suffer from nutrient deprivation, where c-MYC nutrient deprivation signature is high (>50-percentile) in lung, breast, and colon cancers; for lung cancer, the same top tertile was used, while for breast and colon cancers, top 50-percentile was used to select ASS1-high tumors. The effect size was quantified by median survival differences.

### Urea Cycle Dysregulation (UCD) Score

UCD-score quantifies the extent of dysregulation of urea cycle that enhance pyrimidine synthesis. It is defined in a previous study (Lee et al., 2018), as the weighted summation of gene expression of six urea cycle enzymes, i.e., UCD-score = -ASL-ASS1+CPS1-OTC+SLC25A13-SLC25A15, where the names of genes denote their gene expression levels.

### Whole-Exome Sequencing Analysis

4T1 cells treated as outlined above were followed by lysis and DNA isolation using DNeasy blood and tissue kit (Quiagen). Dragen was used to carry out somatic variant calling for the set of samples. The following Dragen filter was used: “SNP filter:snp: QD<2.0 || FS>60.0; indel filter:indel: QD<2.0 || FS>200”, and the resulting variant call was annotated using Ensembl VEP ⁵. Purine to pyrimidine (R>Y) and pyrimidine to purine (Y>R) mutations on the sense strand were counted and pooled per condition to achieve sufficient values for statistical analysis. A Fisher’s exact test was performed on R>Y versus Y>R mutation with background as their expected frequencies from CCLE ⁶ mutation data (file ‘CCLE_hybrid_capture1650_hg19_NoCommonSNPs_NoNeutralVariants_CDS_2012.05.07.ma f’, N=905).

### Association of NDNO Protein Signature and Hydrophobicity of Peptides in CPTAC Lung Cancer Data

The present inventors analyzed recent CPTAC3 lung adenocarcinoma mass spectrometry data (www(dot)cptac-data-portal(dot)georgetown(dot)edu/cptac/s/S046) to evaluate the association of ASS1 over-expression and hydrophobicity of peptides. The mass spectrometry data estimates the presence of peptides in the sample (n=109). Proteomics and peptide repertoire were analyzed for tumor and matched normal samples, to test whether NDNO proteomic signature. Proteomic NDNO score was calculated by getting the regular NDNO score for tumor and matched normal samples following the definition in the Methods and taking the ratio of the NDNO score of tumor over normal sample. The hydrophobicity of peptides was determined by R library ‘Peptides’ using the metric from Janin et al ⁷. The hydrophobicity of each sample’s peptide repertoire was obtained by taking the mean hydrophobicity metric of all peptides present in the given sample. The association between proteomic NDNO-signature and peptide’s hydrophobicity was evaluated using Wilcoxon ranksum test.

### Association of ASS1 Expression and NDNO Signature With Immune Checkpoint Therapy Response

The present inventors first analyzed the association of median ASS1 expression and the objective response rate (ORR) to anti-PD1/PDL1 therapy across cancer types obtained from Dr. Yarchoan ⁸ using Wilcoxon rank-sum test. High ASS1 tumors were determined as the top tertile of ASS1 expression, while the low ASS1 tumors were the remainder of cancer types. Also, the association of median PRB levels was analyzed in each cancer with cross-cancer ORR to anti-PD1/PDL1 therapy in an analogous manner. In difference from the case of ASS1, low PRB tumors were determined as the bottom tertile of PRB levels, while the high PRB tumors were the remainder of cancer types. This is because there are many cancer types whose median PRB levels are zero (or close to zero), and PRB levels are mostly negative. A total of 19 cancer types were considered where TCGA molecular profiles and anti-PD1 ORR data were available for both analyses. The present inventors next analyzed the anti-PD1 treatment cohort of lung cancer patients, where pre-treatment transcriptomic data and response information is available with 7 responders and 14 nonresponders. ASS1 expression levels and NDNO signature were compared between responders vs nonresponders using Wilcoxon ranksum test.

### Identification of Cellular Proteins in NSCLC Tumors

Human tumor samples were collected under the Helsinki protocol MID118-2018. Tumors were meshed into single-cell suspensions and supplemented with 5% SDS, pH 7.5, then heated for 3 min at 95° C. Lysates were then sonicated for 6 cycles (Diagnode). Protein concentration was measured using a BCA assay. Proteins were reduced using 5 mM dithiothreitol and alkylated using iodoacetamide. Samples were then loaded onto an S-trap column (Protifi, USA) and subjected to in-solution tryptic digestion according to the manufacturer’s protocol. The samples were vacuum dried and stored in -80° C. until analysis.

ULC/MS grade solvents were used for all chromatographic steps. Each sample was loaded using split-less nano-Ultra Performance Liquid Chromatography (10 kpsi nanoAcquity; Waters, Milford, MA, USA). The mobile phase was: A) H2O + 0.1% formic acid and B) acetonitrile + 0.1% formic acid. Desalting of the samples was performed online using a reversed-phase Symmetry C18 trapping column (180 µm internal diameter, 20 mm length, 5 µm particle size; Waters). The peptides were then separated using a HSS T3 nano-column (75 µm internal diameter, 250 mm length, 1.8 µm particle size; Waters) at 0.35 µL/min. Peptides were eluted from the column into the mass spectrometer using the following gradient: 4% to 30%B in 163 min, 30% to 90%B in 5 min, maintained at 90% for 5 min and then back to initial conditions. The nanoUPLC was coupled online through a nanoESI emitter (10 µm tip; New Objective; Woburn, MA, USA) to a quadrupole orbitrap mass spectrometer (Q Exactive Plus, Thermo Scientific) using a FlexIon nanospray apparatus (Thermo). Data was acquired in data dependent acquisition (DDA) mode, using a Top10 method. MS1 resolution was set to 70,000 (at 400 m/z), mass range of 300-1650 m/z, AGC of 3e6 and maximum injection time was set to 50 msec. MS2 resolution was set to 17,500, quadrupole isolation 1.7 m/z, AGC of 1e5, dynamic exclusion of 60sec and maximum injection time of 60 msec.

### Mass Spectrometry Data Analysis

Proteins were identified and quantified by the MaxQuant software (version 1.6.0.16 (51)) with default parameters. Masses were searched against the human proteome database from the Uniprot/Swiss-Prot (last update on 9.2018). Using Python 3.6 peptides identified though MaxQuant (50) were initially filtered to remove reverse sequences and known mass spectrometry contaminants. Protein abundances were inferred from peptide intensities using MaxQuant LFQ intensity. Across each sample, a weighted average of amino acid composition was calculated on the basis of the abundance of each protein. The total amino acid composition was then correlated to expression of ASS1 across the same samples using Pearson correlation.

### Metastasis Analysis

To check if ASS1 overexpression is associated with metastatic risk, the present inventors analysed gene expression and metastasis incidence data of 683 breast cancer patients (101 metastasis cases and 582 control) with local/regional recurrence after mastectomy in estimating distant metastasis risk (GSE102484)¹⁴. ASS1 expression levels were compared in these two patient groups using Wilcoxon ranksum test.

### Statistical Analysis

Unless otherwise specified, all statistical analyses were performed using one-way ANOVA, Student’s t-test or Wilcoxon rank sum test of multiple or two groups, with Dunnett’s correction when required. Log-transformed data were used where differences in variance were significant and variances were correlated with means. The sample size was chosen in advance based on common practice of the described experiment and is mentioned for each experiment. Each experiment was conducted with biological and technical replicates and repeated at least three times unless specified otherwise. When samples were distributed non-normally, Mann-Whitney analysis was performed. Statistical tests were done using Statsoft’s STATISTICA, ver. 10. All error bars represent SER. P<0.05 was considered significant in all analyses (* denotes P<0.05, **P<0.01, ***P<0.001).

Data and Software Availability: The raw sequencing data from in vivo experiments will be available through European Nucleotide Archive (www(dot)ebi(dot)ac(dot)uk/ena), and the processed data will be made available through Supplementary Data. The custom code used for TCGA analysis in the revised manuscript is available via github repository at www(dot)github(dot)com/jooslee/ASS1 with github ID: ‘reviewers2019’ and password: ‘github.pass1’.

ShASS1 sequences for the mouse and human cells:

RMM3981-201787524
Clone Id:TRCN0000075720
TACAGTGGCGAACAAATTCAC (SEQ ID NO: 16);

RMM3981-201788578
Clone Id:TRCN0000075719
TTGTAGAGTGAAAGTGGAGAC (SEQ ID NO: 17);

RMM3981-201792294
Clone Id:TRCN0000075718
ATTAATGCTGAAGCCTGGGAG (SEQ ID NO: 18);

RMM3981-201787916
Clone Id:TRCN0000075721
TTGCTTTGCATACTCCATCAG (SEQ ID NO: 19);

RMM3981-201791413
Clone Id:TRCN0000075722
ATAGCCTTGTTCCTTCAGCCA (SEQ ID NO: 20);

RHS3979-200799745-Clone Id: TRCN0000045556
Mature Antisense- ATGATATTCCTTCAGCCTGAG (SEQ ID NO: 21);

RHS3979-200799742
Clone Id-TRCN0000045553
Mature Antisense:-AATGAGCATGGTAAAGGATGG (SEQ ID NO: 22);

### Example 2

### Ass1 Supports Purine Synthesis in Cancer

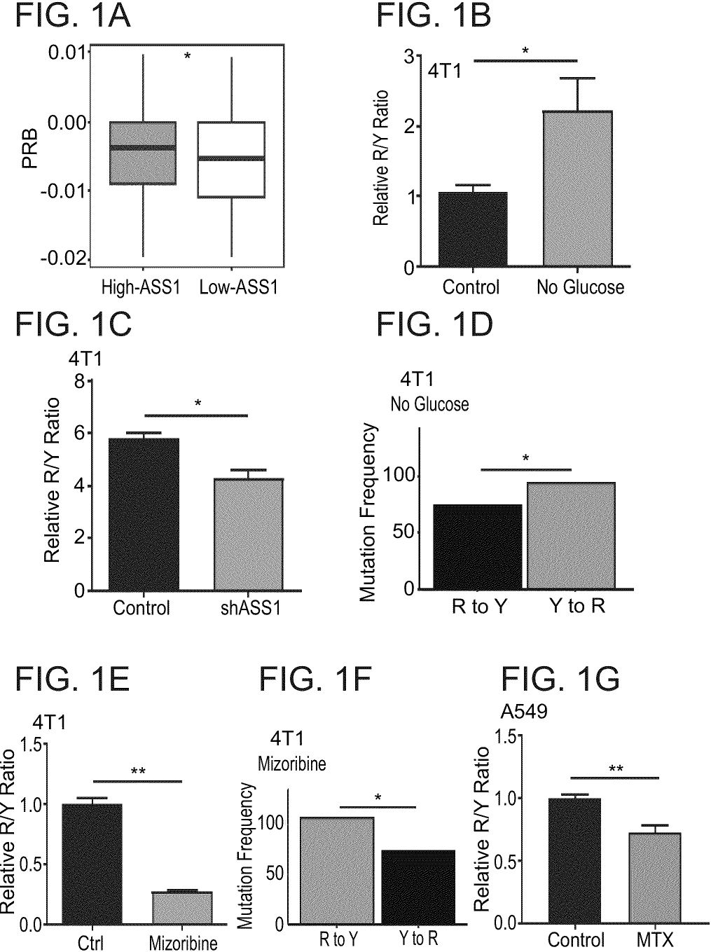

By increasing serine and glycine synthesis, gluconeogenesis flux has been reported to promote purine synthesis in cancer cells¹². Hence it was hypothesized that ASS1-overexpressing tumors might have a reverse, purine-rich mutational bias (PRB), characterized by a higher purine to pyrimidine ratio. Analyzing the TCGA data, it was found that ASS1 levels correlate with a purine rich mutational bias in breast, lung and colorectal cancers collectively (FIG. 1A). Indeed, it was found that in murine breast and colon cancer cells deprived of glucose, purine levels increased, resulting in a significant elevation in the total purine to pyrimidine metabolites’ ratio (FIG. 2A and Table 1). It was confirmed that purine levels decrease in murine breast cancer cells with ASS1 depletion under glucose deprivation (FIG. 1C and Table 2). Furthermore, NO synthesis inhibition decreased the purine to pyrimidine ratio in the murine breast cancer cells with and without ASS1 knockdown (FIG. 2B). Exome sequencing of high ASS 1-expressing cancer cells verified that the high purine to pyrimidine ratio leads to a PRB signature (FIG. 1D). Notably, treatment of these cells with mizoribine, a purine synthesis inhibitor³⁵, reversed their nucleotide imbalance towards higher pyrimidines and resulted in the reversal of the PRB to a pyrimidine-rich mutational signature and in inhibition of′ proliferation (FIGS. 1E-F, FIGS. 2C-D and Table 3). In support, inhibition of one-carbon metabolism by methotrexate treatment in high ASS1 expressing lung cancer cells also decreased their purine to pyrimidine ratio (FIG. 1G and Table 4).

4T1 cells were grown in the absence of glucose for 24 h (baseline) and 48 h and quantified by liquid chromatography-mass spectrometry (LC-MS). Nucleotide levels were measured in µg/ml and corrected for protein concentration.

TABLE 1

Nucleotide levels under normal and low-glucose conditions.

| | Nucleotide | Baseline Level | Low Nutrient Level | p value |
|---|---|---|---|---|
| Purines µg/ml/ pt | AMP | 74.92 | 309.77 | 0.05 |
| | GMP | 63.06 | 392.48 | 0.03 |
| Pyrimidines µg/ml/ pt | UMP | 1.88 | 5.82 | 0.06 |
| | CMP | 15.43 | 29.35 | 0.1 |

4T1 cells expressing either shGFP (control) or shASS1 were grown in the absence of glucose for 48 h and quantified by liquid chromatography-mass spectrometry (LC-MS). Nucleotide levels were measured in µg/ml and corrected for protein concentration.

TABLE 2

Nucleotide levels under normal and low-glucose conditions.

| | Nucleotide | Control Level | shASS1 Level | p value |
|---|---|---|---|---|
| Purines µg/ml/ pt | AMP | 16.96 | 7.53 | 0.02 |
| | GMP | 4.02 | 1.92 | 0.01 |
| Pyrimidines µg/ml/ pt | UMP | 0.3 | 0.23 | 0.37 |
| | CMP | 3.33 | 1.91 | 0.02 |

Nucleotide levels of 4T1 cells grown either with or without 300 µM mizoribine for 24 h, were measured and quantified by liquid chromatography-mass spectrometry (LC-MS) as µg/ml and corrected for protein concentration.

TABLE 3

Nucleotide levels with and without a purine synthesis inhibitor.

| | Nucleotide | Baseline Level | Mizoribine treatment Level | p value |
|---|---|---|---|---|
| Purines µg/ml/ pt | AMP | 991.16 | 483.2 | 0.01 |
| | GMP | 93.1 | 28.46 | 0.004 |
| Pyrimidines µg/ml/ pt | UMP | 21.38 | 38.39 | 0.15 |
| | CMP | 8.74 | 7.3 | 0.7 |

Nucleotide levels of 4T1 cells grown either with or without 1.2 µM Methotrexate (Holland-Moran) for 48 h, were measured and quantified by liquid chromatography-mass spectrometry (LC-MS) as µg/mg protein.

TABLE 4

Nucleotide levels with and without methotrexate.

| | Nucleotide | Baseline Level | Methotrexate Treatment | p value |
|---|---|---|---|---|
| Purines µg/mg pt | AMP | 991.16 | 483.2 | 0.01 |
| | GMP | 93.1 | 28.46 | 0.004 |
| | ATP | 2854.8 | 75.1 | 2.07E-06 |
| | GTP | 60.6 | 16.7 | 0.001 |
| Pyrimidines µg/mg pt | UMP | 21.38 | 38.39 | 0.15 |
| | CMP | 8.74 | 7.3 | 0.7 |
| | TMP | 0.0008 | 0.0078 | 0.24 |
| | UTP | 77.7 | 36.9 | 0.010 |
| | TTP | 0.050808 | 0.004432 | 0.0005 |
| | CTP | 29.4 | 12.0 | 0.002 |

### Example 3

### High ASS1 Cancers Are More Sensitive to Purine Synthesis Inhibition Than Low ASS1-Expressing Tumors

The present inventors have previously demonstrated that the pyrimidine-rich transversion mutational bias observed in low ASS1 tumors enhances sensitivity to anti-PD1 therapy⁹. Thus it is hypothesized that high ASS1 tumors and/or tumors with high PRB levels would be less responsive to immunotherapy. This hypothesis was examined by analyzing the TCGA gene expression data together with the overall response rates to anti-PD1/PDL1 therapy reported for different cancer types³⁶. Though there are outliers, in general it was found that tumors with high ASS1 expression and those with high PRB levels show decreased response for checkpoint therapy compared to other tumors (FIGS. 3A-B). Specifically in NSCLC cohort treated with anti-PD1, it was found that ASS1 expression and NDNO-score are significantly associated with lack of response (FIG. 6C). Taken together, these results testify that high ASS1 expression in nutrient deprived conditions contributes to high PRB levels and poor response to immunotherapy.

Given these findings, the present inventors turned to study their potential translational value regarding treatment of high ASS1 tumors. It was previously shown that high pyrimidine synthesis generates higher levels of hydrophobic peptides that are more immunogenic⁹(Lee et al. supra). Therefore, the protein amino acid composition of NSCLC tumor samples data from the Clinical Proteomic Tumor Analysis Consortium (CPTAC) compendium was analyzed and indeed ASS1 protein expression levels inversely correlate with the abundance of hydrophobic amino acids (FIG. 3D). Since immunoproteasome activity has been associated with enhanced neoantigen presentation and with improved immune responses³⁷, the association of its induced subunit LMP-7^{38,39} with ASS1 levels and with mizoribine treatment was tested. Both interventions are expected to increase immunogenicity by increasing the pyrimidine to purine ratio because mizoribine inhibits purine synthesis, and downregulating ASS1 expression levels with shASS1 is expected to both promote pyrimidine synthesis⁹ as well as decrease purine synthesis. Indeed, it was found that treatment with mizoribine as well as low ASS1 levels are both associated with a higher immunoproteasome LMP-7 expression (FIG. 1E). These results suggest a potential adjuvant benefit for mizoribine treatment of ASS1- high expressing tumors, by promoting the anti-cancer immune response via enhancing immune-proteasome activity. To test the relevance of these findings in vivo, the present inventors had to separate the putative purine inhibitory effects of mizoribine that act directly on the tumor cells from its potential immunosuppressive effects on the tumor microenvironment⁴⁰. Thus, 4T1 breast cancer cells were treated with mizoribine in vitro before injecting them into the mammary pads of immunocompetent mice. While mizoribine and ASS1 inhibition each restricted tumor growth, they generated a more significant response when given in combination with anti-PD1 in breast cancer mice unresponsive to anti-PD1 (FIG. 1F).

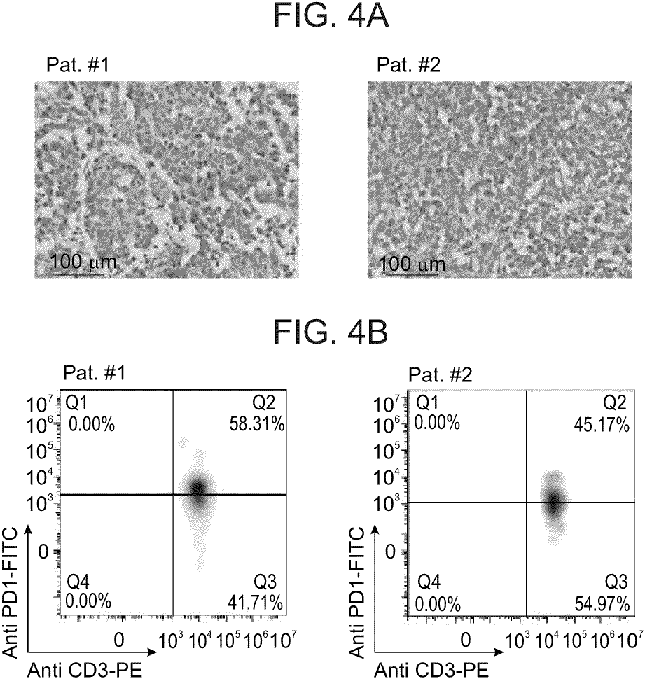

To study the importance of the above results for human tumors on a more mechanistic level, patient derived xenografts (PDX) were grown in immunosuppressed mice. These PDXs were generated from NSCLC explants with high ASS1 expression, taken from cancer patients who were not responding to immune checkpoint therapy (FIG. 4A). Following in vitro treatment with anti-PD1 with or without mizoribine, a significant elevation in secretion of IFN-γ was found by the patients’ autologous primary CD8+ T cells in response to the combined treatments, compared to IFN-γ levels secreted by these cells following treatment with Anti-PD1 alone (FIG. 3G and FIG. 4B). These results together demonstrate that in ASS1 expressing tumors, the purine inhibitor mizoribine can both reverse the high purine to pyrimidine ratio, and enhance the response to immunotherapy.

Since cancer cells that can survive in a low nutrient environment and can evade the immune system have a higher chance to metastasize successfully⁴¹, these findings naturally gave rise to the possibility that high ASS1 expression may contribute to tumor metastatic progression. In support of this notion, others have shown that in gastric cancer, decreasing ASS1 expression can repress metastasis formation⁴². To evaluate whether ASS1 overexpression is associated with metastatic risk, the TCGA data was analyzed for ASS1 expression and metastasis incidence in 683 breast cancer patients (101 metastasis cases and 582 control) with local/regional recurrence after mastectomy. ASS1 levels were significantly higher in primary tumors that developed metastases as compared to primary tumors that did not (FIG. 3H).

### Example 4

### Increasing Pyrimidine to Purine Ratio in Cancer Cells Using Ethyl Protocatechuate (EDHB) or Sildenafil

### Materials and Methods

In-vitro EDHB/Viagra nucleotide analysis: 4T1 cells (600k) were seeded at 10 cm plates. Cells were incubated with 200 uM EDHB (Sigma - 100% Ethanol) for 8 hours. MC38 (600k) and LLC (650 k) were seeded at 10 cm plates and incubated for 24/48 hours with 100 uM of Sildenafil Citrate (Viagra-Focus biomolecules). Subsequently, cells were washed with ice-cold saline, lysed with 50% methanol/ in 5 mM Hepes and quickly scraped followed by three freeze-thaw cycles in liquid nitrogen. The insoluble material was pelleted in a cooled centrifuge (4° C.) and the supernatant was collected for consequent LC-MC analysis. The obtained samples were concentrated in speedvac to eliminate methanol, and then lyophilized to dryness, re-suspended in 200 µl of water and purified on polymeric weak anion columns as follows. Each column was conditioned by passing 1 ml of methanol, then 1 ml of formic acid/methanol/water (2/25/73) and equilibrated with 1 ml of water. The samples were loaded, and each column was washed with 1 ml of water and 1 ml of 50% methanol. The purified samples were eluted with 1 ml of ammonia/methanol/water (2/25/73) followed by 1 ml of ammonia/methanol/water (2/50/50) and then collected, concentrated in speedvac to remove methanol and lyophilized. Before LC-MC analysis, the obtained residues were re-dissolved in 100 µl of water and centrifuged for 5 min at 21,000 g to remove insoluble material.

In-vivo EDHB experiment: Eight-week-old female BALB/c mice were inoculated with 4T1 in the mammary fat pad (1 × 10⁶ cells). ethyl 3,4 dihydroxybenzoate (EDHB) treatment was started after 4 days when the primary tumor nodule was established and palpable. EDHB (40 mg per kg) was intraperitoneally injected daily into cancer-bearing mice. EDHB was initially dissolved in 100% Ethanol and diluted to required concentration in saline. 95% Saline/5% Ethanol mix without drug was injected as a vehicle to control animals. Following injection, mice were treated with 250 ug of anti PD-1 antibody on days 7, 11, 13, 15. On day 19, mice were euthanized and tumors were removed and weighted.

### Generation of PDXs and Patient’s Peripheral CD8+ T Cells

Two patients bearing metastatic non-small cell lung cancer (NSCLC) were included in this study. Blood samples and fresh tumor tissue samples were procured just after their biopsy with patient consent and with Helsinki approval (number 0093-19-SOR). The samples were placed in serum free DMEM (Gibco) media then processed for implantation in NSG (NOD scid gamma) mice (NOD.Cg-Prkdcscid Il2rgtm1Wjl, The Jackson Laboratory). Tumor tissue samples were implanted subcutaneously in dorsal flanks of the 7-9 weeks old male mice to form patient-derived xenografts (PDXs).

All animal experiments were done under Institutional Animal Care and Use Committee (IACUC) of Ben-Gurion University of the Negev (BGU’s IACUC) according to specified protocols aiming to ensure animal welfare and reduce su□ering. The Animal ethical clearance protocol number used for this research is IL-80-12-2015.

After isolation of peripheral blood mononuclear cells (PBMCs) from the peripheral blood of the patients according to standard protocol using Lymphocyte Separation Medium (LSM, MP Biomedicals, SKU 0850494-CF), CD8⁺ T cells were isolated from PBMCs by employing human CD8 Microbeads (Miltenyi Biotec, 130-045-201), LS column (Miltenyi Biotec, 130-042-401) and MidiMACS Separator (130-042-302) following manufacturer’s protocol. For culturing and maintenance of CD8 T cells, RPMI containing 10% human male AB plasma (Sigma, H4522), 1 mM sodium pyruvate, 2 mM L-glutamine, 0.1 mM MEM non-essential amino acids, 1% penicillin/streptomycin, 10 mM HEPES (Life Technologies), 200 IU/mL recombinant human IL-2 (PeproTech) and 50 ng/mL anti-human CD3 Antibody (BioLegend, 317302) were used for the first 48 hrs followed by culturing and passaging in media containing 200 IU/mL of IL-2.

### Results

FIGS. 6A-C show that Ethyl 3,4 dihydroxybenzoate (EDHB) increases pyrimidine to purine and potentiates efficacy of anti PD-1 treatment in vivo. FIG. 6A - Ethyl 3,4 dihydroxybenzoate (EDHB) treatment increases cellular deoxypyrimidine to deoxypurine ratio. 600,000 4T1 breast cancer cells were plated in 10 cm dishes and incubated with 200 uM EDHB for 8 hours. Nucleotide levels were quantified by liquid chromatography-mass spectrometry (LC-MS). The graph demonstrates an increase in pyrimidine / purine ratio following EDHB treatment. FIG. 6B - Ethyl 3,4 dihydroxybenzoate (EDHB) and anti PD-1 combination treatment reduces tumor weight in-vivo. Female Balb/c mice were inoculated with 4T1 cells into the mammary fat pad. After 4 days of tumor initiation, mice were treated daily with 40 mg/kg EDHB or saline administrated IP. 250 ug of anti PD-1 was administrated IP at days 7, 11, 13 and 15. Tumor weight was measured following sacrifice. FIG. 6C - Treatment did not cause any change in the total weight of the mice.

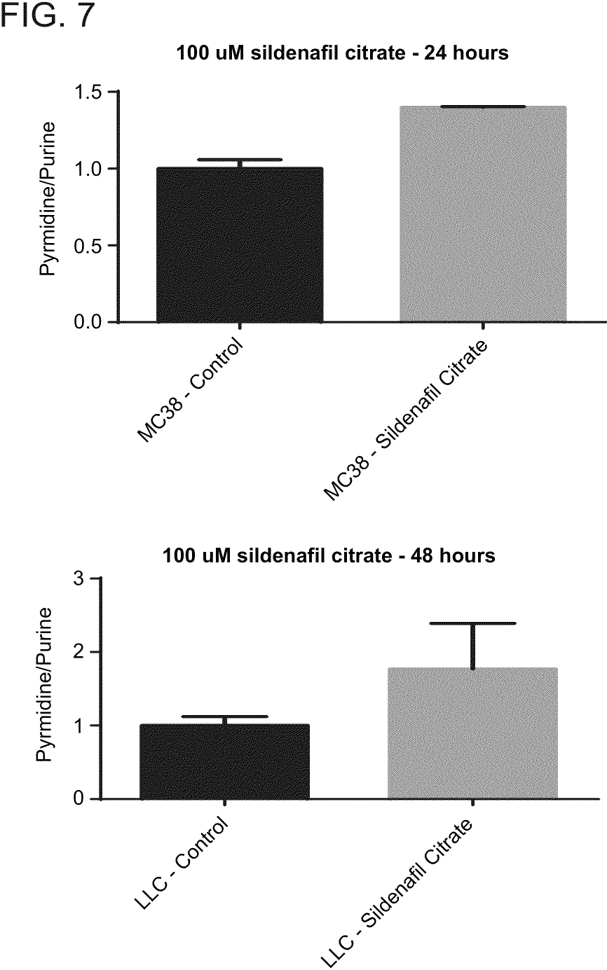

FIG. 7 shows that Sildenafil Citrate (Viagra®) treatment increases cellular pyrimidine to purine ratio. 600,000 mc-38 mouse colon cancer cells (left panel) and 650,000 LLC mouse lung cancer cells (right panel), were plated in 10 cm dishes and incubated with 100 uM Sildenafil citrate for 24/48 hours respectively. Nucleotide levels were and quantified by liquid chromatography-mass spectrometry (LC-MS).

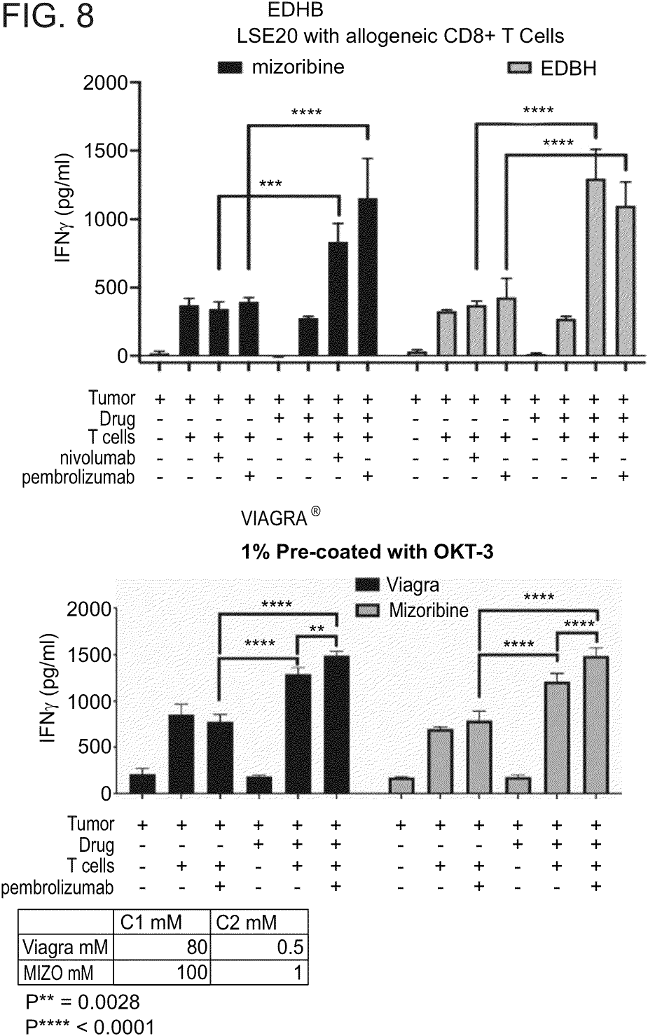

FIG. 8 shows that Sildenafil Citrate (Viagra) and EDHB treatments increases response to anti-PD1 therapy in PDX of non responsive patients

PDXs from patients with lung cancer non responsive to anti-PD1 were exposed to no treatment, to mizoribine, or to either EDHB (left panel) or Viagra (right panel). IFN-g response was measured following treatment with anti-PD1.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Specification

1 Husson, A., Brasse-Lagnel, C., Fairand, A., Renouf, S. & Lavoinne, A. Argininosuccinate synthetase from the urea cycle to the citrulline-NO cycle. Eur J Biochem 270, 1887-1899 (2003).

2 Erez, A., Nagamani, S. C. & Lee, B. Argininosuccinate lyase deficiency-argininosuccinic aciduria and beyond. Am J Med Genet C Semin Med Genet 157, 45-53 (2011).

3 Liu, Q. et al. Reduced expression of argininosuccinate synthetase 1 has a negative prognostic impact in patients with pancreatic ductal adenocarcinoma. PLoS One 12, e0171985, doi:10.1371/journal.pone.0171985 (2017).

4 Sahu, D. et al. Argininosuccinate Synthetase 1 Loss in Invasive Bladder Cancer Regulates Survival through General Control Nonderepressible 2 Kinase-Mediated Eukaryotic Initiation Factor 2alpha Activity and Is Targetable by Pegylated Arginine Deiminase. Am J Pathol, doi:10.1016/j.ajpath.2016.09.004 (2016).

5 Kim, Y. et al. Reduced argininosuccinate synthetase expression in refractory sarcomas: Impacts on therapeutic potential and drug resistance. Oncotarget 7, 70832-70844, doi:10.18632/oncotarget.12225 (2016).

6 Wu, L. et al. Expression of argininosuccinate synthetase in patients with hepatocellular carcinoma. J Gastroenterol Hepatol 28, 365-368, doi:10.1111/jgh.12043 (2013).

7 Rabinovich, S. et al. Diversion of aspartate in ASS1-deficient tumours fosters de novo pyrimidine synthesis. Nature 527, 379-383, doi:10.1038/nature15529 (2015).

8 Kim, J. et al. CPS1 maintains pyrimidine pools and DNA synthesis in KRAS/LKB1-mutant lung cancer cells. Nature 546, 168-172, doi:10.1038/nature22359 (2017).

9 Lee, J. S. et al. Urea Cycle Dysregulation Generates Clinically Relevant Genomic and Biochemical Signatures. Cell 174, 1559-1570 e1522, doi:10.1016/j.cell.2018.07.019 (2018).

10 Delage, B. et al. Arginine deprivation and argininosuccinate synthetase expression in the treatment of cancer. Int J Cancer 126, 2762-2772 (2010).

11 Szlosarek, P. W. et al. Aberrant regulation of argininosuccinate synthetase by TNF-alpha in human epithelial ovarian cancer. International journal of cancer. Journal international du cancer 121, 6-11, doi:10.1002/ijc.22666 (2007).

12 Vincent, E. E. et al. Mitochondrial Phosphoenolpyruvate Carboxykinase Regulates Metabolic Adaptation and Enables Glucose-Independent Tumor Growth. Mol Cell 60, 195-207, doi:10.1016/j.molcel.2015.08.013 (2015).

13 Mesonero, J., Matosin, M., Cambier, D., Rodriguez-Yoldi, M. J. & Brot-Laroche, E. Sugar-dependent expression of the fructose transporter GLUT5 in Caco-2 cells. Biochem J 312 (Pt 3), 757-762, doi:10.1042/bj3120757 (1995).

14 Bean, G. R. et al. A metabolic synthetic lethal strategy with arginine deprivation and chloroquine leads to cell death in ASS1-deficient sarcomas. Cell Death Dis 7, e2406, doi:10.1038/cddis.2016.232 (2016).

15 Bateman, L. A. et al. Argininosuccinate Synthase 1 is a Metabolic Regulator of Colorectal Cancer Pathogenicity. ACS Chem Biol 12, 905-911, doi:10.1021/acschembio.6b01158 (2017).

16 Miles, L., Heubi, J. E. & Bove, K. E. Hepatocyte glycogen accumulation in patients undergoing dietary management of urea cycle defects mimics storage disease. Journal of pediatric gastroenterology and nutrition 40, 471-476 (2005).

17 Kuhara, H. et al. Neonatal type of argininosuccinate synthetase deficiency. Report of two cases with autopsy findings. Acta pathologica japonica 35, 995-1006 (1985).

18 Bigot, A., Tchan, M. C., Thoreau, B., Blasco, H. & Maillot, F. Liver involvement in urea cycle disorders: a review of the literature. J Inherit Metab Dis 40, 757-769, doi:10.1007/s10545-017-0088-5 (2017).

19 Postic, C. et al. Dual roles for glucokinase in glucose homeostasis as determined by liver and pancreatic beta cell-specific gene knock-outs using Cre recombinase. The Journal of biological chemistry 274, 305-315 (1999).

20 Perez, C. J. et al. Two Hypomorphic Alleles of Mouse Ass1 as a New Animal Model of Citrullinemia Type I and Other Hyperammonemic Syndromes. Am J Pathol (2010).

21 Pennisi, P. et al. Recombinant human insulin-like growth factor-I treatment inhibits gluconeogenesis in a transgenic mouse model of type 2 diabetes mellitus. Endocrinology 147, 2619-2630, doi:10.1210/en.2005-1556 (2006).

22 Rashed, H. M., Nair, B. G. & Patel, T. B. Regulation of hepatic glycolysis and gluconeogenesis by atrial natriuretic peptide. Arch Biochem Biophys 298, 640-645, doi:10.1016/0003-9861(92)90460-e (1992).

23 Zareba-Koziol, M., Szwajda, A., Dadlez, M., Wyslouch-Cieszynska, A. & Lalowski, M. Global analysis of S-nitrosylation sites in the wild type (APP) transgenic mouse brain-clues for synaptic pathology. Molecular & cellular proteomics : MCP 13, 2288-2305, doi:10.1074/mcp.M113.036079 (2014).

24 Pae, H. O. et al. Nitric oxide protects murine embryonic liver cells (BNL CL.2) from cytotoxicity induced by glucose deprivation. Pharmacology & toxicology 86, 140-144 (2000).

25 Cherry, P. D. & Wolin, M. S. Ascorbate activates soluble guanylate cyclase via H2O2-metabolism by catalase. Free Radic Biol Med 7, 485-490 (1989).

26 Keilhoff, G., Reiser, M., Stanarius, A., Aoki, E. & Wolf, G. Citrulline immunohistochemistry for demonstration of NOS activity in vivo and in vitro. Nitric Oxide 4, 343-353, doi:10.1006/niox.2000.0298 (2000).

27 Park, S. W. et al. Nitric oxide upregulates the cyclooxygenase-2 expression through the cAMP-response element in its promoter in several cancer cell lines. Oncogene 24, 6689-6698, doi:10.1038/sj.onc.1208816 (2005).

28 Sun, L. et al. cMyc-mediated activation of serine biosynthesis pathway is critical for cancer progression under nutrient deprivation conditions. Cell Res 25, 429-444, doi:10.1038/cr.2015.33 (2015).

29 Tsai, W. B. et al. Resistance to arginine deiminase treatment in melanoma cells is associated with induced argininosuccinate synthetase expression involving c-Myc/HIF-1alpha/Sp4. Mol Cancer Ther 8, 3223-3233 (2009).

30 Follis, A. V., Hammoudeh, D. I., Wang, H., Prochownik, E. V. & Metallo, S. J. Structural rationale for the coupled binding and unfolding of the c-Myc oncoprotein by small molecules. Chemistry & biology 15, 1149-1155, doi:10.1016/j.chembiol.2008.09.011 (2008).

31 Erez, A. et al. Requirement of argininosuccinate lyase for systemic nitric oxide production. Nat Med 17, 1619-1626 (2011).

32 Consortium, E. P. An integrated encyclopedia of DNA elements in the human genome. Nature 489, 57-74, doi:10.1038/nature11247 (2012).

33 Walz, S. et al. Activation and repression by oncogenic MYC shape tumour-specific gene expression profiles. Nature 511, 483-487, doi:10.1038/nature13473 (2014).

34 Burrows, N. et al. Hypoxia-induced nitric oxide production and tumour perfusion is inhibited by pegylated arginine deiminase (ADI-PEG20). Sci Rep 6, 22950, doi:10.1038/srep22950 (2016).

35 Yokota, S. Mizoribine: mode of action and effects in clinical use. Pediatr Int 44, 196-198, doi:10.1046/j.1328-8067.2002.01536.x (2002).

36 Yarchoan, M., Hopkins, A. & Jaffee, E. M. Tumor Mutational Burden and Response Rate to PD-1 Inhibition. The New England journal of medicine 377, 2500-2501, doi:10.1056/NEJMc1713444 (2017).

37 Spits, M. & Neefjes, J. Immunoproteasomes and immunotherapy-a smoking gun for lung cancer? J Thorac Dis 8, E558-563, doi:10.21037/jtd.2016.05.21 (2016).

38 Basler, M., Kirk, C. J. & Groettrup, M. The immunoproteasome in antigen processing and other immunological functions. Curr Opin Immunol 25, 74-80, doi:10.1016/j.coi.2012.11.004 (2013).

39 Lee, M. et al. Expression of Immunoproteasome Subunit LMP7 in Breast Cancer and Its Association with Immune-Related Markers. Cancer Res Treat 51, 80-89, doi:10.4143/crt.2017.500 (2019).

40 Vijayan, D., Young, A., Teng, M. W. L. & Smyth, M. J. Targeting immunosuppressive adenosine in cancer. Nat Rev Cancer 17, 709-724, doi:10.1038/nrc.2017.86 (2017).

41 Fu, Y., Liu, S., Zeng, S. & Shen, H. The critical roles of activated stellate cells-mediated paracrine signaling, metabolism and onco-immunology in pancreatic ductal adenocarcinoma. Mol Cancer 17, 62, doi:10.1186/s12943-018-0815-z (2018).

42 Shan, Y. S. et al. Argininosuccinate synthetase 1 suppression and arginine restriction inhibit cell migration in gastric cancer cell lines. Sci Rep 5, 9783, doi:10.1038/srep09783 (2015).

43 Montal, E. D. et al. PEPCK Coordinates the Regulation of Central Carbon Metabolism to Promote Cancer Cell Growth. Molecular cell 60, 571-583, doi:10.1016/j.molcel.2015.09.025 (2015).

44 Possemato, R. et al. Functional genomics reveal that the serine synthesis pathway is essential in breast cancer. Nature 476, 346-350, doi:10.1038/nature10350 (2011).

45 Antonov, A. et al. Bioinformatics analysis of the serine and glycine pathway in cancer cells. Oncotarget 5, 11004-11013, doi:10.18632/oncotarget.2668 (2014).

46 DeBerardinis, R. J. & Chandel, N. S. Fundamentals of cancer metabolism. Sci Adv 2, e1600200, doi:10.1126/sciadv.1600200 (2016).

47 Erb, N., Harms, D. O. & Janka-Schaub, G. Pharmacokinetics and metabolism of thiopurines in children with acute lymphoblastic leukemia receiving 6-thioguanine versus 6-mercaptopurine. Cancer chemotherapy and pharmacology 42, 266-272, doi:10.1007/s002800050816 (1998).

What is claimed is:

1. A method of treating a solid tumor in a subject in need thereof, the method comprising administering to the subject a therapeutically effective amount of an agent which increases pyrimidine to purine ratio in a cell without decreasing pyrimidine synthesis, thereby treating the solid tumor.

2. (canceled)

3. The method of claim 1, wherein said agent is not anti-folate.

4. The method of claim 1, wherein said agent decreases purine synthesis.

5. The method of claim 4, wherein said agent comprises mizoribine or derivative thereof.

6. The method of claim 1, wherein said agent does not affect purine synthesis.

7. The method of claim 6, wherein said agent enhances pyrimidine synthesis.

8. The method of claim 6, wherein said agent comprises ethyl protocatechuate (EDHB).

9. The method of claim 6, wherein said agent enhances purine cyclization.

10. The method of claim 9 wherein said agent is selected from the group consisting of BRL-50481 and Sildenafil (e.g., Viagra®).

11. The method of claim 10, wherein said solid tumor is non-responsive to an immune-modulating drug.

12. The method of claim 11, further comprising administering to the subject an immune-modulating drug.

13-14. (canceled)

15. The method of claim 12, wherein said agent is administered prior to said immune-modulating drug.

16. The method of claim 15, wherein said solid tumor is metastatic.

17. The method of claim 16, wherein said solid tumor is selected from the group consisting of lung cancer, breast cancer and colon cancer.

18. The method of claim 17, wherein said lung cancer is non-small cell lung cancer (NSCLC).

19. The method of claim 18, wherein said solid tumor is characterized by Purine-Rich mutational Bias (PRB).

20. The method of claim 19, wherein said immune-modulating drug is an immune-check point protein modulator.

21. The method of claim 20, wherein said an immune-check point protein modulator is a PD1 antagonist.
